(19) United States
(12) Patent Application Publication
(10) Pub. No.: US 2026/0263596 A1
(43) Pub. Date: Sep. 10, 2026

(54) Programming B Cells to Produce IL-27 to Treat Inflammatory Diseases

(71) Applicant: Board of Regents, The University of Texas System, Austin, TX (US)

(72) Inventors: Zhengming Xu, San Antonio, TX (US); Shuai Wu, San Antonio, TX (US); Hui Yan, San Antonio, TX (US); Weiwei Luo, San Antonio, TX (US)

(21) Appl. No.: 18/994,949

(22) PCT Filed: Aug. 17, 2023
(86) PCT No.: PCT/US2023/030417

§ 371 Date: Jan. 15, 2025

Related U.S. Application Data
(60) Provisional application No. 63/399,021, filed on Aug. 18, 2022.

(51) Int. Cl. A61K 40/13; A61K 40/35; C12N 5/0781

(52) U.S. Cl. CPC A61K 40/13; A61K 40/35; C12N 5/0635; C12N 2500/40; C12N 2501/599

(57) ABSTRACT

Embodiments of the invention are directed to B cells programmed in vitro ex vivo to robustly produce a pleiotropic cytokine IL-27 as well as methods for making and using the programmed B cells. The reprogramming process use naive B cells that are primed by an innate stimulus, including toll-like receptor ligands (TLRs) (TLR1/2, TLR3, TLR4, TLR5, TLR7, TLR8 and TLR9) and active cyclic dinucleotides (CDNs), and optionally stimulated with CD 154 and IL-21. The unique conditioning of IL-27-producing B cells (referred to as B-27 cells hereafter) places B-27 cells at the intersection of innate and adaptive immune regulation of the gut.

### RELATED APPLICATION

This application is an International Application claiming priority to U.S. Provisional Application 63/399,021 filed Aug. 18, 2022 which is incorporated herein by reference in its entirety.

### STATEMENT REGARDING FEDERALLY FUNDED RESEARCH

None

### FIELD OF THE INVENTION

The invention is directed generally to the field of medicine and cell-based therapy. Particular embodiments are directed to methods of in vitro programming of B cells for production of IL-27 and methods of using the resulting programmed IL-27 producing B cells for therapy.

### BACKGROUND

Inflammatory Bowel Disease (IBD) refers to a group of gastrointestinal disorders characterized by a chronic non-specific inflammation of portions of the gastrointestinal tract. Ulcerative colitis (UC) and Crohn's Disease (CD) are the most prominent examples of IBD in humans. They are associated with many symptoms and complications, including growth retardation in children, rectal prolapse, blood in stools (e.g., melena and/or hematochezia), wasting, iron deficiency, and anemia (e.g., iron deficiency anemia and anemia of chronic disease or of chronic inflammation).

The etiology (or etiologies) and pathogenesis of IBD are still unclear. Previous understanding of the pathogenesis was limited to a three-stage process: (a) an irritant, which could be an immune process or infectious agent, (b) activation of leukocytes which release enzymes such as pro-inflammatory cytokines (particularly tumor necrosis factor alpha (TNF-α), proteases, and inflammatory mediators such as histamine, serotonin and prostaglandins, and (c) induction of edema, pain, heat and loss of function (See, e.g., Wyngaarden and Smith (eds.) Cecil's Textbook of Medicine (W. B. Saunders Co. 1985), Berkow (ed.); The Merck Manual of Diagnosis and Therapy (Merck Sharp & Dohme Research Laboratories, 1982); Harrison's Principles of Internal Medicine, 12th Ed., McGraw-Hill, Inc. (1991)).

Numerous theories implicate multiple factors leading up to IBD including genetic predisposition, environmental factors, infectious agents, and immunologic alterations (See e.g., Kirsner, J. B., et al. (eds), Inflammatory Bowel Disease, 3rd ed., Lea and Febiger, Philadelphia (1988); Zipser, R. D., (ed.), Dig. Dis. Sci., 33 Suppl.:1S-87S (1988)). The immunologic alterations in IBD appear to be autoimmune in nature, with autoantibodies and lymphocyte-cytotoxicity directed against intestinal epithelial cells. However, even the latest developments in the immunologic aspects of the pathogenesis of IBD cannot answer the basic question of whether the detected changes in humoral and cellular immunity reflect a primary defect or secondary response to injury.

Treatment for IBD currently includes steroids, sulphasalazine and its derivatives, and immunosuppressive drugs (e.g., cyclosporin A, mercaptopurine and azathioprine). Such therapies are directed toward suppression of the general immune response. These approaches may result in poor success and have little or no selectivity. These approaches can be accompanied by unwanted and sometimes dangerous consequential side effects.

There is a need for additional compositions and therapeutic approaches for treating IBD and related diseases.

### SUMMARY

The Inventors have discovered novel methods and compositions to address the problems associated with treating IBD described above. Embodiments of the invention are directed to B cells programmed in vitro ex vivo to robustly produce a pleiotropic cytokine IL-27, which is a heterodimer of IL-27p28 and EBI3, as well as methods for making and using the programmed B cells. The reprogramming process use naïve B cells that are primed by an innate stimulus, including ligands of any of the 7 toll-like receptor agonists/ligands (TLRs) (TLR1/2, TLR3, TLR4, TLR5, TLR7, TLR8 and TLR9) and active cyclic dinucleotides (CDNs), and optionally stimulated with CD154 and/or IL-21. The unique conditioning of IL-27-producing B cells (referred to as B-27 cells hereafter) places B-27 cells at the intersection of innate and adaptive immune regulation of the gut.

Certain embodiments are directed to methods for treating inflammatory bowel disease (IBD) comprising: (i) obtaining naïve B cells from a compatible subject; (ii) culturing the naïve B cells in the presence of a toll-like receptor (TLR) agonist and cyclic dinucleotide or a mimic thereof resulting in or forming an IL-27 secreting B cell (B-27 cell); and (iii) administering the B27 cell to a subject having IBD or suspected of having IBD. In certain aspects the naïve B cells are isolated from the subject to be treated. The naïve B cells can be isolated from the blood. In certain aspects the naïve B cells are isolated from peripheral blood mononuclear cells (PBMC). In certain aspects the TLR agonist is a TLR 1/2, TLR 4, TLR 7, TLR 9 agonist or a combination thereof. In certain aspects the TLR 1/2 agonist is PAM2CSK4. In other aspects the TLR 4 agonist is lipid A. In still other aspects the TLR 7 agonist is R-848. In certain aspects the TLR 9 agonist is a CpG oligodeoxynucleotide (ODN). The TLR 9 agonist can be an ODN having the nucleotide sequence of (CpG ODN1826: 5′-tccatgacgttcctgacgtt-3′ (20 mer, SEQ ID NO:1) with a full phosphorothiolate backbone or CpG ODN 2006: 5′-tcgtcgttttgtegttttgtcgit-3′ (24 mer, SEQ ID NO:2). In certain aspects 1×10⁴ to 1×10⁸ B-27 cells are administered. In still other aspects the B-27 cells are administered intravenously.

Certain embodiments are directed to programmed B cells comprising an IL-27 secreting B cell produced by exposing a naïve B cell to IL-27 programming conditions, the programming conditions consisting essentially of exposure to a toll-like receptor (TLR) agonist and cyclic dinucleotide. The cyclic dinucleotide can be cGAMP. The TLR agonist can be CpG oligodeoxynucleotide or lipopolysaccharide (LPS).

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of in vitro programming of Naïve B cells.

FIG. 2A-2F. Data demonstrating Il27p28 gene transcription, as quantified by GFP signals from Tg(Il27p28-Gfp) reporter mice, and an important role of B-27 cells in gut homeostasis. (A) Imaging of gut and mesenteric lymph node (MLN) showing GFP+ cells primarily in the ileum of non-DSS-treated Tg(Il27p28-Gfp) mice; (B) Flow cytometry analysis of the proportions of different immune cell types within GFP+ leukocytes (CD45+) in gut lymphoid tissues in Tg(Il27p28-Gfp) mice treated with DSS (2%; 7 d); (C) Body weight and composite colitis disease activity index (DAI; occult bleeding, 0-6, and stool consistency, 0-6) of μMT:Il27p28−/− and μMT:Il27p28+/+ mice fed with DSS (2%) in drinking water for 7 d; (D) μMT:Il27p28−/− and μMT:Il27p28+/+ mice were treated with DSS for 7 d and sacrificed at d 10 for analysis of gut histology and colon length; (E, F) Same analyses as in (C) and (D), respectively, but involved μMT:Ebi3−/− mice and “wildtype” μMT:Ebi3+/+ mice.

FIG. 3A-3D. Role of CDNs, STING, cGAS and commensal bacteria in B-27 cell induction and/or gut homeostasis. (A) Il27p28, Ebi3 and Il12a expression and IL-27 secretion by purified B cells after priming with nil, cGAMP (30 μg/ml) for 1 h or LPS (3 μg/ml) for 24 h and stimulated with nil, CD154 (top panels) or αCD40 (bottom) plus IL-21 for 24 h. (B) IL-27 secretion by B cells after priming with different CDNs (all 30 μg/ml), as indicated, for 1 h and stimulated with αCD40 plus IL-21 for 48 h. (C) IL-27 secretion by B cells after priming with cGAMP and LPS at different dose combinations, for 24 h and then stimulated with αCD40 plus IL-21 for 24 h. (D) μMT:Sting−/− and μMT:Sting+/+ mice were treated with DSS (7 d) and sacrificed at d 10 to analyze IL-27 production and gut damages.

FIG. 4A-4E. B-27 cells impact differentiation of ILC3s. (A, B) Intracellular staining/FACS analysis of ILC1s (GATA3-RORγt-T-bet+), ILC2s (GATA3+RORγt−), total ILC3s (GATA3-RORγt+), as well as the ratio of the two different subsets of ILC3s (T-bet+NKp46+ and T-bet−/loNKp46−/lo) in different gut tissues isolated from mixed bone marrow chimera μMT:Ebi3−/− mice and their wildtype counterparts after DSS treatment (2%; 7 d). A representative gating strategy (A, left) and intracellular staining of IL-22 in the two ILC3 subsets (A, right) are also shown. (C) qRT-PCR analysis of selected cytokine genes in the same mice/tissues as in (A, B). (D) ELISA of IgM and IgA in the feces of μMT:Ebi3−/− mice and their wildtype counterparts before (nil) and after DSS treatment, as indicated. (E) FACS analysis of different CD4+ T cell subsets (IFNγ+ Th1, IL-4+ Th2, IL-17A+ Th17 and FoxP3+ Treg cells) in the same tissues as in (A, B). T cells from small intestine and colon were difficult to recover after culturing with GolgiSTOP for cytokine intracellular staining.

FIG. 5A-5C. IL-27 polarizes ILC3s to express T-bet and IL-22. ILC3s were sort enriched (70%) by surface markers (IL-7R+CD45+Lin−CD117+ST2−) from mesenteric lymph nodes and cultured in the presence of IL-2 and IL-7 (to promote ILC survival) as well as IL-1B plus IL-23 (to promote ILC3 proliferation)—CD117 and ST2 are preferentially, albeit not exclusively, expressed by ILC3s and ILC2s, respectively. After 7 d of culture, ILCs were amplified by >10-fold and accounted for >95% of all cells. After washing to remove cytokines, ILC3s were stimulated with nil, IL-1P/IL-23 (to promote basal IL-22 expression in ILC3s) or IL-27 for 12 h. Cells were analyzed by FACS for expression of (A) T-bet, NKp46 and (B) IL-22, IL-17A and IFNγ, as quantified in (C).

FIG. 6A-6H. B-27 cells target ILC3s to maintain gut homeostasis. (A, B) body weight loss (A) and disease index (B) of mice with deficiency of IL-27 receptor specifically in cells that expressed RORγt, which is encoded by the gene Rorc and is a hallmark transcription factor of ILC3s and Th17 cells (Rorc^{cre/+}Il27ra^{fl/fl}), and their wildtype littermates (Rorc^{+/+}Il27ra^{fl/fl}) after treatment with DSS. (C). Survival curve of the mice in (A, B). Mice were euthanized after losing more than 25% of body weight or showed moribund signs. (D, E). colon length (D) and immunohistochemistry analysis of gut inflammation (E) in the same mice as in (A-C). (F, G). body weight loss (F) and disease index (G) of mixed bone marrow chimera mice with T cell-specific deficiency of IL-27 receptor specifically in T cells (Tcr^{-/-}:Il27ra^{-/-}), including Th17 cells, and their wildtype mouse counterparts (Tcr^{-/-}:Il27ra^{+/+}) after treatment with DSS. H. colon length in the same mice as in (F, G).

FIG. 7A-7E. Analysis of huB-27 cells in IBD patients. (A) immunofluorescence staining of IL-27 (red) and B cell marker CD20 (green) in four ILFs in a UC patient. (B-D) immunohistochemistry of IL-27 and CD20 in healthy controls (HCs, car accident deaths; n=7, 1 female), UC (n=6, 3 females) and CD (n=34, 7 females) patients and quantification of total and IL-27+ ILFs. (E) Illustration of biopsy sites, i.e., rectum (Site 1), Sigmoid colon(Site 2), Descending colon (Site 3), Transverse colon (Site 4), Ascending colon (Site 5) and terminal ileum (TI; Site 6). UC patients show varied inflammation in Site 1-5 but generally not in Site 6, and CD patients can have colon or TI inflammation.

FIG. 8A-8C. Programmed B-27 cells reduce gut damages. (A) C57 mice were injected i.v. with 10\hat{ }6 purified naïve B cells or B-27 cells (after LPS/CD154/IL-21 co-stimulation for 48 h) every other day, starting one day before DSS (2% in drinking water) treatment. Body weight was monitored until d 13. Each arrow indicates one injection. (B, C) C57 mice were injected i.v. with 10\hat{ }6 purified WT or B-27-KO B cells co-stimulated with LPS/CD154/IL-21 for 48 h, using the same regimen as that in (A). Body weight and DAI were monitored daily until d 8 (B), when mice were sacrificed to analyze colon length and gut histology (C).

FIG. 9. Immune cell population #1 (CD19⁺FAS⁺PD-L1⁺IL-27p28⁺ B cells), #7 (PD-L1+ T cells), #10 (CD3⁺CD4⁺IL-7Ra⁺CXCR3⁺IL-27p28⁺ T cells), #11 (Ly6G+ neutrophils), #13 (CD3+CD4+ T cells) and #14 (CD3+CD11c+Il7Rα+\RORγt+ T cells) were disturbed by the DSS treatment, but were rescued by B-27 cells. Other immune cell populations are: #2 (CD19⁺RORγt⁻PD-L1⁺ B cells), #3 (CD19⁺ B cells), #4 (CD19⁺IL-7Ra⁺PD-L1⁺ B cells), #5 (CD19⁺IL-7Rα⁺PD-L1⁺ B cells), #6 (IL-7Ra⁺RORγt+NCR⁺PD-L1⁺, ILCs), #8 (IL-7Rα+CD11b⁺CD11c⁺RORγt⁺PD-L1⁺, macrophages), #9 (IL-7Rα⁺CD11c⁺T-bet⁺); #12 (CD3⁺CD11c⁺IFNγ⁺T-bet⁺CXCR3⁺, T effector cells); #13 (CD3⁺CD4⁺, T cells), #15 (IgA⁺FAS⁺CD11c⁻CD11b⁺, switched B cells); #16 (CD3⁺CD4IL-7Rα⁺PD-L1⁺IL-27p28⁺NCR⁺), #17 (CD11b⁺CD11c⁺FAS⁺IL-7Rα⁺PD-L1⁺IgA⁺) and #18 (IL-7Rα⁺RORγt⁺CXCR3⁺IgA⁺).

FIG. 10. CpG and cGAMP synergize to induce IL-27 production.

FIG. 11. B-27 cells can be generated by priming naïve B cells with yeast cell wall components and then stimulated with CD154 and IL-21. Secretion of IL-27 (top) and CXCL10 (bottom) by B cells first stimulated with β-glucan, mannan or zymosan, all from Saccharomyces cerevisiae, for 4 hours followed by the addition of CD154 and IL-21, both of which are Tfh cell ligands, for 48 hours.

FIG. 12. Tracking of in vitro programmed B-27 cells in recipient mice by congenic markers. C57 recipient mice, which expressed the CD45.2 isoform of the CD45 gene, were injected with in vitro programmed B-27 cells (5×10⁶ cells/injection) once every two days, from day −1 to day 5, with DSS injected at day 0. Such B-27 donor cells were produced by isolating naïve B cells from either CD45.2⁺ C57 mice (left two columns) or CD45.1⁺ C57 mice (right two columns), priming them with LPS for 24 hours and then stimulating them with anti-CD40 mAb plus IL-21 for 24 hours. Injected recipient mice were euthanized at day 14 for flow cytometry analysis of the proportion of donor and host B cells in the B cell compartment of different tissues and organs, as indicated.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

Cell-based therapeutics (CBTs) have emerged as highly specific and effective therapies for immune diseases and are projected to be “the next pillar of medicine” (Science Translational Medicine, 2013, 5: 179ps7). For example, the chimeric-antigen receptor T cell (CAR-T) therapy has been approved by FDA to treat leukemia and is currently being developed to treat different types of solid tumors. One significant advantage of CBTs over small molecule compounds and biologics (such as monoclonal antibodies) is that patients' own cells are reprogrammed to obtain therapeutic effects and, therefore, well-tolerated by the body, causing minimal rejection of the transplanted cells or damage to the host tissues/organs. CBTs also have durable effects, owing to their homing to and survival in certain niches as well as acquisition of proliferative potentials in selected cases, including CAR-T. Embodiments of the current invention are directed to programmed B-27 cells and their use in cell-based therapeutics.

Homeostasis in the lower gastrointestinal tract is maintained by multiple interactions among the host immune system, microbiota (commensal bacteria, fungi, and viruses), and neuronal system elements. An obligatory cause (and likely consequence too) of such complex interactions is the functional dichotomy of many elements. For instance, commensal bacteria generate not only dietary metabolites that control inflammation, such as short-chain fatty acid butyrate, but also compounds that promote gut inflammation. Even more complicated are functions of cytokines, particularly those showing both pro-inflammatory and anti-inflammatory activities in multiple target cell types. It has been known that T cells play a critical role in maintaining the gut immune balance, e.g., through anti-inflammatory Tregs and pathogenic Th17 cells. Innate lymphoid cells (ILCs), which function through cytokine production in a manner similar to T cell counterparts, also orchestrate gut homeostasis, e.g., through reciprocal regulation with T cells. In particular, the two subsets of Group 3 ILCs (ILC3s), i.e., pro-wound healing and IL-22-producing T-bet⁺NKp46⁺ ILC3s and pro-inflammatory and IL-17-producing T-bet^{-/lo}NKp46^{-/lo}ILC3s are inter-convertible, and their ratio dictates the immune balance.

In addition, B cells are present in many segments of the gastrointestinal tract, including gut-associated lymphoid tissues (GALTs). Studies on the role of B cells in the gut are extensive, but largely focused on T-dependent and T-independent production of IgA Abs, which sculpt the microbiota and prevent commensal bacteria from activating intestinal epithelial cells. In addition to differentiating into Ab-secreting plasma cells as “B effector cells”, B cells can function as “regulatory cells” by producing cytokines, such as IL-10, TGF-β and IL-35, many of which are implicated in gut homeostasis.

Embodiments of the invention are directed to B cells programmed in vitro or ex vivo to robustly produce cytokine IL-27. The reprogramming process use naïve B cells that are primed by an innate stimulus, including ligands of any of the 7 Toll-Like Receptor Ligands (TLRs) (TLR1/2, TLR3, TLR4, TLR5, TLR7, TLR8 and TLR9) and active cyclic dinucleotides (CDNs), and optionally stimulated with CD154 and IL-21, the hallmark factors of T follicular helper (Tfh) cells. Owing to their unique induction condition B-27 cells are at the intersection of innate and adaptive immune regulation of the gut. Indeed, mice with B cell-specific knockout (KO) in Il27p28 or Ebi3 (IL-27 negative mice) display increased wasting disease and colitis upon treatment of chemical dextran sulfate sodium (DSS). Such B-27 cell-deficient mice show marked reduction in T-bet⁺NKp46⁺ ILC3s, and concomitant increase in pathogenic T-bet^{-/lo}NKp46^{-/lo}ILC3 subset of innate lymphoid cell (ILC). Accordingly, IL-27 can polarize ILC3s in vitro to express T-bet and IL-22. The in vitro programmed B-27 cells, when adoptively transferred into C57 mice, completely protect mice from DSS-triggered colonic damage and prevent colitis development. By contrast, Il27p28 KO B cells reprogrammed the same way fail to prevent gut damage.

As shown in humans, few isolated lymphoid follicles (ILFs) exist in healthy subjects. In patients with ulcerative colitis (UC) and Crohn's disease (CD), the two most common types of inflammatory bowel diseases (IBDs), the number of ILFs is increased, up to 20 ILFs per examined section of the GI tract. Such ILFs show IL-27 expression, which occurs largely within CD20+ B cells suggesting that B cell production of IL-27 is among the host response to gut injuries, thereby providing the rationale to use adoptively transferred human B-27 cells to control gut inflammation. Indeed, human naïve B cells can be primed with TLR9 ligand CpG optionally followed by stimulation with CD154 and IL-21 to upregulate IL27p28 and EBI3. Like mouse IL-27, human IL-27 can activate STAT transcription factors, indicating that reprogrammed human B-27 cells can be potential therapeutics for inflammatory diseases, including IBDs.

Toll-Like Receptor (TLR) Agonist. Toll-like receptors (TLRs) are the best characterized of the pattern recognition receptors (PRRs) (Ishii et al., 2008). They are highly conserved transmembrane proteins, consisting of an ectodomain with multiple leucine-rich repeats for pattern recognition, a membrane-spanning α-helix, and a Toll/interleukin-1 receptor (TIR) domain for intracellular signaling. At least 13 mammalian TLRs have been identified, each specifically localizing to either the plasma membrane or endosomal membranes, and each detects a unique complement of pathogen associated molecular patterns (PAMPs)(Akira et al., 2006; Shi et al., 2006).

A TLR agonist is any compound or substance that functions to activate a TLR, e.g., to induce a signaling event mediated by a TLR signal transduction pathway. Suitable TLR agonists include TLR1 agonists, TLR2 agonists, TLR3 agonists, TLR4 agonists, TLR5 agonists, TLR6 agonists, TLR7 agonists, TLR8 agonists, and TLR9 agonists.

The term “agonist,” as used herein, refers to a compound that can combine with a receptor (e.g., a TLR) to produce a cellular activity. An agonist may be a ligand that directly binds to the receptor. Alternatively, an agonist may combine with a receptor indirectly by, for example, forming a complex with another molecule that directly binds to the receptor. An agonist may be referred to as an agonist of a particular TLR (e.g., a TLR7 agonist) or a particular combination of TLRs (e.g., a TLR 7/8 agonist—an agonist of both TLR7 and TLR8).

The terms “CpG-ODN,” “CpG nucleic acid,” “CpG polynucleotide,” and “CpG oligonucleotide,” used interchangeably herein, refer to a polynucleotide that comprises at least one 5′-CG-3′ moiety, and in many embodiments comprises an unmethylated 5′-CG-3′ moiety. In general, a CpG nucleic acid is a single- or double-stranded DNA or RNA polynucleotide having at least six nucleotide bases that may comprise, or consist of, a modified nucleotide or a sequence of modified nucleosides. In some embodiments, the 5′-CG-3′ moiety of the CpG nucleic acid is part of a palindromic nucleotide sequence. In some embodiments, the 5′-CG-3′ moiety of the CpG nucleic acid is part of a non-palindromic nucleotide sequence.

TLR2 agonists include isolated, naturally-occurring TLR2 agonists and/or synthetic TLR2 agonists. TLR2 agonists include synthetic triacylated and diacylated lipopeptides. A non-limiting example of a TLR2 ligand is FSL-1 (a synthetic lipoprotein derived from Mycoplasma salivarium 1), Pam₃Cys (tripalmitoyl-S-glyceryl cysteine) or S-[2,3-bis(palmitoyloxy)-(2RS)-propyl]-N-palmitoyl-(R)-cysteine, where “Pam₃” is “tripalmitoyl-S-glyceryl”) (Aliprantis et al., 1999). Derivatives of Pam₃Cys are also suitable TLR2 agonists, where derivatives include, but are not limited to, S-[2,3-bis(palmitoyloxy)-(2-R,S)-propyl]-N-palmitoyl-(R)-Cys-(S)-Ser-(Lys)4-hydroxytrihydrochloride; Pam₃Cys-Ser-Ser-Asn-Ala; PaM₃Cys-Ser-(Lys)₄; Pam₃Cys-Ala-Gly; Pam₃Cys-Ser-Gly; Pam₃Cys-Ser; PaM₃Cys-OMe; Pam₃Cys-OH; PamCAG, palmitoyl-Cys((RS)-2,3-di(palmitoyloxy)-propyl)-Ala-Gly-OH; and the like. Another non-limiting example of a suitable TLR2 agonist is Pam₂CSK₄ PaM₂CSK₄ (dipalmitoyl-S-glyceryl cysteine-serine-(lysine)₄; or Pam₂Cys-Ser-(Lys)₄) is a synthetic diacylated lipopeptide. Synthetic TLRs agonists have been described in the literature. See, e.g., Kellner et al. (1992); Seifer et al. (1990); Lee et al. (2003).

TLR3 agonists include isolated, naturally-occurring TLR3 agonists and/or synthetic TLR3 agonists. TLR3 agonists include naturally-occurring double-stranded RNA (dsRNA); synthetic ds RNA; and synthetic dsRNA analogs; and the like (Alexopoulou et al., 2001). An exemplary, non-limiting example of a synthetic ds RNA analog is poly(I:C).

Suitable TLR4 agonists include isolated, naturally-occurring TLR4 agonists and/or synthetic TLR4 agonists. TLR4 agonists include naturally-occurring lipopolysaccharides (LPS), e.g., LPS from a wide variety of Gram negative bacteria; derivatives of naturally-occurring LPS; synthetic LPS; bacteria heat shock protein-60 (Hsp60); mannuronic acid polymers; flavolipins; teichuronic acids; S. pneumoniae pneumolysin; bacterial fimbriae, respiratory syncytial virus coat protein; and the like. TLR4 agonist also include monophosphoryl lipid A-synthetic (MPLAs, Invivogen) and Phosphorylated HexaAcyl Disaccharide (PHAD, Avanti Polar Lipids), as well as other synthetic TLR4 agonists.

Suitable TLR5 agonists include isolated, naturally-occurring TLR5 agonists and/or synthetic TLR5 agonists. TLR5 agonists include but are not limited to highly conserved 22 amino acid segment of flagellin as well as full length flagellin and other segments thereof.

Suitable TLR7 agonists include isolated, naturally-occurring TLR7 agonists and/or synthetic TLR7 agonists. TLR7 ligands include imidazoquinoline compounds; guanosine analogs; pyrimidinone compounds such as bropirimine and bropirimine analogs; and the like.

Suitable TLR8 agonists include isolated, naturally-occurring TLR8 agonists and/or synthetic TLR8 agonists. TLR8 agonists include, but are not limited to, compounds such as R-848

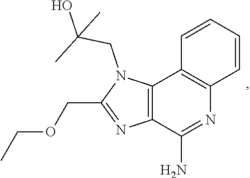 [SMILES: CCOCc1nc2c(N)nc3ccccc3c2n1CC(C)(C)O ; ","]

and derivatives and analogs thereof. Suitable TLR8 agonists include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, and 6-, 7-, 8-, or 9-aryl or heteroaryl substituted imidazoquinoline amines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

Suitable TLR9 agonists include isolated, naturally-occurring TLR9 agonists and/or synthetic TLR9 agonists. Examples of TLR9 agonists (also referred to herein as “TLR9 ligands”) include nucleic acids comprising the sequence 5′-CG-3′ (a “CpG nucleic acid”), in certain aspects C is unmethylated. The terms “polynucleotide,” and “nucleic acid,” as used interchangeably herein in the context of TLR9 ligand molecules, refer to a polynucleotide of any length, and encompasses, inter alia, single- and double-stranded oligonucleotides (including deoxyribonucleotides, ribonucleotides, or both), modified oligonucleotides, and oligonucleosides, alone or as part of a larger nucleic acid construct, or as part of a conjugate with a non-nucleic acid molecule such as a polypeptide. Thus, a TLR9 ligand may be, for example, single-stranded DNA (ssDNA), double-stranded DNA (dsDNA), single-stranded RNA (ssRNA) or double-stranded RNA (dsRNA). TLR9 ligands also encompasses crude, detoxified bacterial (e.g., mycobacterial) RNA or DNA, as well as enriched plasmids enriched for a TLR9 ligand. In some embodiments, a “TLR9 ligand-enriched plasmid” refers to a linear or circular plasmid that comprises or is engineered to comprise a greater number of CpG motifs than normally found in mammalian DNA.

In some embodiments, a TLR9 ligand for use in a subject method is an oligonucleotide, e.g., consists of a sequence of from about 5 nucleotides to about 200 nucleotides, from about 10 nucleotides to about 100 nucleotides, from about 12 nucleotides to about 50 nucleotides, from about 15 nucleotides to about 25 nucleotides, from 20 nucleotides to about 30 nucleotides, from about 5 nucleotides to about 15 nucleotides, from about 5 nucleotides to about 10 nucleotides, or from about 5 nucleotides to about 7 nucleotides in length. In some embodiments, a TLR9 ligand that is less than about 15 nucleotides, less than about 12 nucleotides, less than about 10 nucleotides, or less than about 8 nucleotides in length is associated with a larger molecule.

In some embodiments, a TLR9 ligand does not provide for expression of a peptide or polypeptide in a eukaryotic cell, e.g., introduction of a TLR9 ligand into a eukaryotic cell does not result in production of a peptide or polypeptide, because the TLR9 ligand does not provide for transcription of an mRNA encoding a peptide or polypeptide. In these embodiments, a TLR9 ligand lacks promoter regions and other control elements necessary for transcription in a eukaryotic cell.

In general, a TLR9 ligand used in a subject composition comprises at least one unmethylated CpG motif. The relative position of any CpG sequence in a polynucleotide in certain mammalian species (e.g., rodents) is 5′-CG-3′(i.e., the C is in the 5′ position with respect to the G in the 3′ position).

In other embodiments, a TLR9 ligand comprises one or more TCG sequences at or near the 5′ end of the nucleic acid; and at least two additional CG dinucleotides. In some of these embodiments, the at least two additional CG dinucleotides are spaced three nucleotides, two nucleotides, or one nucleotide apart. In some of these embodiments, the at least two additional CG dinucleotides are contiguous with one another. In some of these embodiments, the TLR9 ligand comprises (TCG)n, where n=1 to 3, at the 5′ end of the nucleic acid. In other embodiments, the TLR9 ligand comprises (TCG)n, where n=1 to 3, and where the (TCG)n sequence is flanked by one nucleotide, two nucleotides, three nucleotides, four nucleotides, or five nucleotides, on the 5′ end of the (TCG)n sequence.

The core structure of a TLR9 ligand useful in the invention may be flanked upstream and/or downstream by any number or composition of nucleotides or nucleosides. In some embodiments, the core sequence of a TLR9 ligand is at least 6 bases or 8 bases in length, and the complete TLR9 ligand (core sequences plus flanking sequences 5′, 3′ or both) is usually between 6 bases or 8 bases, and up to about 200 bases in length.

A TLR9 ligand useful in carrying out a subject method can comprise one or more of any of the above CpG motifs. For example, a TLR9 ligand useful in the invention can comprise a single instance or multiple instances (e.g., 2, 3, 4, 5 or more) of the same CpG motif. Alternatively, a TLR9 ligand can comprise multiple CpG motifs (e.g., 2, 3, 4, 5 or more) where at least two of the multiple CpG motifs have different consensus sequences, or where all CpG motifs in the TLR9 ligand have different consensus sequences.

Cyclic Dinucleotides (CDNs). A cyclic dinucleotide “CDN” refers to a cyclic dinuculeotide such as 3′-5′ c-di-AMP, 3′-5′ c-di-GMP, 3′-3′ cGAMP (also known as 3′-5′,3′-5′cGAMP), or 2′-3′ cGAMP (also known as 2′-5′,3′-5′ cGAMP). Type I IFNs, produced both by innate immune cells in the tumor microenvironment and by the tumor cells themselves, are known to mediate anti-tumor effects against several malignancies, due to their ability to intervene in all phases of cancer immune-editing. (Zitvogel et al., Nat Rev Immunol. 2015, 15:405-14). STING (stimulator of interferon genes), is a major regulator of Type I IFN innate immune responses to pathogens, following recognition of cytosolic DNA by the sensor cyclic GMP-AMP synthase (cGAS). cGAS catalyzes the synthesis of cyclic GMP-AMP (cGAMP), which in turn functions as a second messenger that binds and activates STING. (Zhao et al., Biochim Biophys Acta. 2015, 1852:365-78. PMID: 24807060). The term “STING agonist” refers to a molecule that binds to STING (stimulator of interferon genes, or TMEM173), activates it, and triggers activation of the IRF3-TBK1 pathway leading to increased transcription of type 1 interferon and other genes.

### I. B CELL THERAPEUTICS

For effective antibody responses to infectious and environmental antigens, specific B cells recognize arrayed epitopes that crosslink their B cell receptors (BCRs) and later need to be engaged by CD154 (CD40 ligand, CD40L) expressed on cognate T helper (Th) 35 cells for robust proliferation and differentiation in germinal centers (GCs), from which plasma cells emerge to secrete mature antibodies. These are of switched Ig isotypes (IgG, IgA, and IgE) and high affinity, as underpinned by Ig locus class switch DNA recombination (CSR) and somatic hypermutation (SHM), respectively. Prior to the T-dependent adaptive phase, both specific and bystander B cells have already been exposed to antigen- or host cell-derived stimuli that engage innate receptors, particularly Toll-like receptors (TLRs). Accordingly, engagement of B-cell TLR can synergize with BCR crosslinking to induce AID, a DNA deaminase essential for CSR and SHM, and elicit class-switched and high-affinity IgG antibodies during the T-independent phase of antibody responses or stand-alone T-independent responses. In immune-competent subjects, TLR ligands can function as adjuvants to potentiate antibody responses, partially by priming dendritic cells (DCs) that in turn activate Th cells. However, the relevance of TLR priming to B cell activation by T cell stimuli in the T-dependent antibody response remains unclear.

Independent from their differentiation into antibody-secreting plasma cells, B cells are known to produce cytokines (Sonnenberg and Artis, Nat. Med. 21, 698-708 (2015); Yan et al., bioRxiv doi. 10.1101/2020.06.26.117010 (2020); Troy et al., J. Immunol. 183, 2037-2044 (2009); Sasaoka et al., AJP: Gastrointestinal and Liver Physiology 300, G568-G576 (2011)), including those that direct CSR from IgM to specific Ig isotypes, such as IL-4 (IgG1), TGFb (IgA) and IFNg (murine IgG2a), thereby supplementing cytokine production by Th cells. In addition, mouse B cells can be induced to produce cytokines IL-10 and IL-35 (heterodimer of Il12a-encoded IL12a and Ebi3-encoded EBI3), thereby acting as immunosuppressive B regulatory (Breg) cells in several pathophysiological conditions (Tato et al., PLoS One 7, e31680 (2012); Diegelmann et al., J Biol. Chem. 287, 286-298 (2012)). The Inventors explore new regulatory functions of B cells, starting with profiling cytokine gene induction in B cells by innate stimuli TLR ligands in combination with T cell stimuli, thereby mimicking B cell activation during the antibody response.

Collecting Naïve B cells. Peripherial Blood Mononuclear Cells (PBMCs) can be isolated from the blood of healthy human donors by density separation over Lymphoprep™ (Axis-Shield, Oslo, Norway) or by using other known methods. Total B-cells can be isolated, for example, from human PBMCs by negative selection using a magnetic sorting device (Miltenyi Biotec, Auburn, Calif.). Briefly, PBMCs can be incubated with a cocktail of biotin-conjugated antibodies, followed by microbead-conjugated anti-biotin Abs for magnetic depletion. B-cells can be eluted according to the manufacturer's protocols.

### II. B-27 TREATMENT FOR INFLAMMATORY BOWEL DISEASES (IBDS)

The current treatment for Inflammatory Bowel Diseases (IBDs) (including UC and CD) includes corticosteroids, anti-inflammatory agents, tumor necrosis factor inhibitors, immunosuppressants, antibiotics, Alpha 4 Integrin inhibitors, and anti-diarrheal agents. All of which have well-documented side effects. New therapeutic approaches are needed. Like CAR-T, IL-27-producing B cells, as programmed in vitro using a patient's own B cells, can be injected intravenously (i.v.) back into the patient's blood stream. They home into inflammatory sites, particularly the gut, to secrete IL-27, which would in turn act on a host of immune cells to dampen inflammation, either directly as an anti-inflammatory cytokine or indirectly by induing the production of other anti-inflammatory cytokines. Commercially, programmed IL-27-producing B cells can potentially be either monotherapy or in combination with other therapeutics in treating IBDs, such as UC and CD. The cases of these two types of IBD have risen quickly, with each affecting more than 30,000 new patients each year and almost 1 million in total in U.S. IBD patients may potentially benefit from our new CBT approach.

B-27 cell compositions may be used in methods of treating subjects having an inflammatory disease or condition. Such adoptive transfer of B-27 cells can be effective to suppress a wide variety of diseases, including, but not limited to autoimmune diseases, inflammatory diseases, or any other disease which may be treated by introduction of a B-27 cell population into a subject. Adoptive transfer of B-27 cells can further be employed to treat IBDs.

In an example of an adoptive transfer protocol, a mixed population of cells is initially extracted from a target donor. Suitably, the B cells are selected (naïve B cells) from the subject. The cells isolated from the donor may be isolated from any location in the donor in which they reside including, but not limited to, the blood, spleen, lymph nodes, and/or bone marrow of the donor. In certain aspects the cells can be obtained from the subject, expanded, and/or programmed, and returned to the subject.

Harvested lymphocytes may be separated by flow cytometry or other cell separation techniques based on B cell markers and then transfused to a recipient. Alternatively, the cells may be stored for future use. In one aspect of this embodiment, the donor and the recipient are the same. In another aspect of this embodiment, the donor is a subject other than the recipient. In a further aspect of this embodiment, the “donor” comprises multiple donors other than the recipient, wherein the naïve B cells from said multiple donors are pooled. The B cells obtained from the donor(s) can be expanded. The cells are programmed or conditioned ex vivo to produce elevated levels of IL-27 as described herein prior to being administered to a recipient.

In the methods of using the programmed B cells contemplated herein, the donor is a subject other than the recipient and the recipient and the donor are histocompatible. Histocompatibility is the property of having the same or mostly the same alleles of major histocompatibility complex (MHC) genes. These genes are expressed in most tissues as antigens. When transplanted cells and/or tissues are rejected by a recipient, the bulk of the immune system response is initiated through the MHC proteins. MHC proteins are involved in the presentation of foreign antigens to T cells and receptors on the surface of the T cell are uniquely suited to recognition of proteins of this type. MHCs are highly variable between individuals and therefore the T cells from the host may recognize the foreign MHC with a very high frequency leading to powerful immune responses that cause rejection of transplanted tissue. When the recipient and the donor are histocompatible the chance of rejection of the B-27 cell population by the recipient is minimized.

The amount or number of B-27 cells which will be effective in the treatment and/or suppression of a disease or disorder which may be treated by introduction of a B-27 cell population into a subject can be determined by standard clinical techniques. The dosage or number of cells will depend on the type of disease to be treated, the severity and course of the disease, the composition being administered, the purpose of introducing the B-27 cell population, previous therapy the recipient has undertaken, the recipient's clinical history and current condition, and the discretion of the attending physician. For example, the specific dose for a particular subject depends on age, body weight, general state of health, diet, the timing and mode of administration, the rate of excretion, medicaments used in combination and the severity of the disorder to which the therapy is applied. Dosages for a given patient can be determined using conventional considerations, e.g., by customary comparison of the differential activities of the compositions of the invention, such as by means of an appropriate conventional pharmacological or prophylactic protocol. The number of cells administered in the composition may also be determined empirically.

The B-27 cell population can be administered in a treatment regime consistent with the disease, e.g., a single or a few doses over one to several days to ameliorate a disease state or periodic doses over an extended time to inhibit disease progression and prevent disease recurrence. For example, the composition may be administered two or more times separated by 4 hours, 6 hours, 8 hours, 12 hours, a day, two days, three days, four days, one week, two weeks, or by three or more weeks. The precise dose to be employed in the formulation will also depend on the route of administration, the seriousness of the disease or disorder, and whether the disease is chronic in nature and should be decided according to the judgment of the practitioner and each patient's circumstances.

The maximal dosage for a subject is the highest dosage that does not cause undesirable or intolerable side effects. The number of variables related to an individual prophylactic or treatment regimen are large and a considerable range of doses can be employed given specific circumstances. The route of administration will also impact the dosage requirements. It is anticipated that dosages of the compositions will reduce symptoms of the condition at least 10%, 20%, 30%, 40%, 50%, 60%, 70%, 80%, 90%/o or 100% compared to pre-treatment symptoms or symptoms left untreated. It is specifically contemplated that pharmaceutical preparations and compositions may palliate or alleviate symptoms of the disease without providing a cure, or in some embodiments may be used to cure the disease or disorder. Effective doses may be extrapolated from dose-response curves derived from in vitro or animal model test systems. Exemplary, non-limiting doses that could be used in the treatment of human subjects range from at least 4×10⁴, at least 4×10⁵, at least 4×10⁶, at least 4×10⁷, at least 4×10⁸, at least 4×10⁹, or at least 4×10¹⁰ B cells/m² including all values and ranges there between. In a certain embodiment, the dose used in the treatment of human subjects ranges from or form about 4×10⁸ to about 4×10¹⁰B cell/m².

For use in the methods described herein, the compositions may be administered by any means known to those skilled in the art, including, but not limited to, intraperitoneal, parenteral, intravenous, intramuscular, subcutaneous, or intrathecal. Thus, the compositions may suitably be formulated as an injectable formulation.

Compositions described herein can be administered to treat gastrointestinal inflammation, chronic gastrointestinal inflammation, chronic gastrointestinal inflammatory conditions, inflammatory bowel disease, and similar conditions.

“Gastrointestinal inflammation” as used herein refers to inflammation of a mucosal layer of the gastrointestinal tract and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation can also be characterized by periods of spontaneous remission and spontaneous occurrence. “Mucosal layer of the gastrointestinal tract” is meant to include mucosa of the bowel (including the small intestine and large intestine), rectum, stomach (gastric) lining, oral cavity, and the like.

“Chronic gastrointestinal inflammation” refers to inflammation of the mucosa of the gastrointestinal tract that is characterized by a relatively longer period of onset, is long-lasting (e.g., from several days, weeks, months, or years and up to the life of the subject) and is associated with infiltration or influx of mononuclear cells and can be further associated with periods of spontaneous remission and spontaneous occurrence. Thus, subjects with chronic gastrointestinal inflammation may be expected to require a long period of supervision, observation, or care. “Chronic gastrointestinal inflammatory conditions” (also referred to as “chronic gastrointestinal inflammatory diseases”) having such chronic inflammation include, but are not necessarily limited to, inflammatory bowel disease (IBD), colitis induced by environmental insults (e.g., gastrointestinal inflammation (e.g., colitis) caused by or associated with (e.g., as a side effect) a therapeutic regimen, such as administration of chemotherapy, radiation therapy, and the like), colitis in conditions such as chronic granulomatous disease (Schappi et al., Arch. Dis. Child., 1984:147, 2001), celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammation caused by an infectious agent, and other like conditions.

Acute and chronic inflammation is secondary to an increase in pro-inflammatory cytokines (particularly tumor necrosis factor-alpha) and an increase in epithelial cell apoptosis. The resultant manifestations of these factors are a loss of the mucosal epithelial lining and the above stated neutrophil/monocyte infiltrate.

As used herein, “inflammatory bowel disease” or “IBD” refers to any of a variety of diseases characterized by inflammation of all or part of the intestines. Examples of inflammatory bowel disease include, but are not limited to, Crohn's disease, ulcerative colitis, irritable bowel syndrome, mucositis, radiation induced enteritis, short bowel syndrome, celiac disease, colitis, stomach ulcers, diverticulitis, pouchitis, proctitis, and chronic diarrhea. Reference to IBD throughout the specification is often referred to in the specification as exemplary of gastrointestinal inflammatory conditions and is not meant to be limiting.

As used herein, the term “symptoms of IBD” is herein defined to detected symptoms such as abdominal pain, diarrhea, rectal bleeding, weight loss, fever, loss of appetite, and other more serious complications, such as dehydration, anemia and malnutrition. Symptoms are subject to quantitative analysis (e.g., weight loss, fever, anemia, etc.). Some symptoms are readily determined from a blood test (e.g., anemia) or a test that detects the presence of blood (e.g., rectal bleeding). The term “wherein said symptoms are reduced” refers to a qualitative or quantitative reduction in detectable symptoms, including but not limited to a detectable impact on the rate of recovery from disease (e.g., rate of weight gain). The diagnosis is typically determined by way of an endoscopic observation of the mucosa, and pathologic examination of endoscopic biopsy specimens.

As used herein, the term “a therapeutically effective amount” of a composition comprising B-27 cells is herein defined as the dosage level required for a subject such that the subject's symptoms of IBD are reduced.

As used herein, the phrase “under conditions such that the symptoms are reduced” refers to any degree of qualitative or quantitative reduction in detectable symptoms of IBD, including but not limited to, a detectable impact on the rate of recovery from disease (e.g., rate of weight gain), or the reduction of at least one of the following symptoms: abdominal pain, diarrhea, rectal bleeding, weight loss, fever, loss of appetite, dehydration, anemia, distention, fibrosis, inflamed intestines and malnutrition.

As used herein, the term “at risk for IBD” is herein defined as encompassing the segment of the world population that has an increased risk (i.e., over the average person) for IBD and can occur at any age. It occurs worldwide, but is most common in the United States, England, and northern Europe. It is especially common in people of Jewish descent. An increased frequency of this condition has been recently observed in developing nations. Increased risk is also most prevalent in people with family members who suffer from inflammatory bowel disease.

As used herein, the term “therapeutic composition comprising B-27 cells” refers to compositions containing B-27 cells together with one or more compounds or agents including, but not limited to, ACE inhibitors, other therapeutic agents, physiologically tolerable liquids, gels, carriers, diluents, adjuvants, excipients, salicylates, steroids, immunosuppressants, antibodies, cytokines, antibiotics, binders, fillers, preservatives, stabilizing agents, emulsifiers, and buffers.

As used herein, the terms “host,” “subject” and “patient” refer to any animal, including but not limited to, human and non-human animals (e.g., rodents, non-human primates, ovines, bovines, ruminants, lagomorphs, porcines, caprines, equines, canines, felines, aves, etc.), that is studied, analyzed, tested, diagnosed or treated. As used herein, the terms “host,” “subject” and “patient” are used interchangeably.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “effective amount” refers to the amount of a composition (e.g., comprising B-27 cells) sufficient to effect beneficial or desired results. An effective amount can be administered in one or more administrations, applications or dosages and is not intended to be limited to a particular formulation or administration route.

As used herein, the terms “administration” and “administering” refer to the act of giving a therapeutic treatment (e.g., compositions of the present invention) to a subject (e.g., a subject or in vivo, in vitro, or ex vivo cells, tissues, and organs). Routes of administration to the human body can be by injection (e.g., intravenously, subcutaneously, intratumorally, intraperitoneally, etc.) and the like.

As used herein, the terms “co-administration” and “co-administering” refer to the administration of at least two agent(s) (e.g., composition comprising a B-27 cell and one or more other agents—e.g., a steroid) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” or grammatical equivalents encompasses the improvement and/or reversal of the symptoms of disease (e.g., inflammatory bowel disease). A composition which causes an improvement in any parameter associated with disease may thereby be identified as a therapeutic composition. The term “treatment” refers to both therapeutic treatment and prophylactic or preventative measures. For example, those who may benefit from treatment with compositions and methods of the present invention include those already with a disease and/or disorder (e.g., inflammatory bowel disease) as well as those in which a disease and/or disorder is to be prevented or ameliorated (e.g., using a prophylactic treatment of the present invention).

As used herein, the term “at risk for disease” refers to a subject (e.g., a human) that is predisposed to experiencing a particular disease. This predisposition may be genetic (e.g., a particular genetic tendency to experience the disease, such as heritable disorders), or due to other factors (e.g., environmental conditions, exposures to detrimental compounds present in the environment, etc.). Thus, it is not intended that the present invention be limited to any particular risk, nor is it intended that the present invention be limited to any particular disease.

As used herein, the term “suffering from disease” refers to a subject (e.g., a human) that is experiencing a particular disease. It is not intended that the present invention be limited to any particular signs or symptoms, nor disease. Thus, it is intended that the present invention encompass subjects that are experiencing any range of disease (e.g., from sub-clinical manifestation to full-blown disease) wherein the subject exhibits at least some of the indicia (e.g., signs and symptoms) associated with the particular disease.

As used herein, the terms “disease” and “pathological condition” are used interchangeably to describe a state, signs, and/or symptoms that are associated with any impairment of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors.

As used herein, a “sufficient amount” or “an amount sufficient to” achieve a particular result refers to a number of B 27 cells of the invention that is effective to produce a desired effect, which is optionally a therapeutic effect (i.e., by administration of a therapeutically effective amount). For example, a “sufficient amount” or “an amount sufficient to” can be an amount that is effective to alter the severity of the subject's condition.

A “therapeutically effective” amount as used herein is an amount that provides some improvement or benefit to the subject. Alternatively stated, a “therapeutically effective” amount is an amount that provides some alleviation, mitigation and/or decrease in at least one clinical symptom. Clinical symptoms associated with disorders that can be treated by the methods of the invention are well-known to those skilled in the art. Further, those skilled in the art will appreciate that the therapeutic effects need not be complete or curative, as long as some benefit is provided to the subject. It is likely that the “therapeutically effective” number of cells required to “treat” an individual will depend on the source of the B cells, the immunological status of the patient at time of blood harvest, the condition of the individual at the time of treatment, and the level of therapeutic treatment with immunosuppressive drugs or agents at the time of treatment as well-known to those skilled in the art.

Various types of inflammatory disease can be treated in accordance with the methods described herein. Non-limiting examples of inflammatory diseases include, but are not limited to, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, after reading the present disclosure, appreciate that many changes can be made in the specific embodiments which are disclosed and still obtain a like or similar result without departing from the spirit and scope of the invention.

### Example 1—B-27 Cells and their Use for Treating IBDs

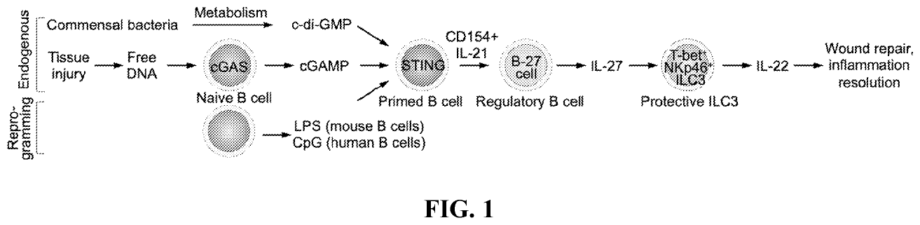

In addition to differentiating into plasma cells that secrete microbiota-shaping protective IgA antibodies, B cells may function as regulatory cells that are induced to produce cytokines to maintain gut integrity. In mouse gut-associated lymphoid tissues and mesenteric lymph nodes, B cells were the primary source of IL-27 in homeostasis and upon induction by dextran sulfate sodium (DSS)-elicited intestinal injury. Production of pleiotropic cytokine IL-27 (EBI3/p28) by B cells was dependent on their expression of STING, consistent with the ability of cGAMP, the cyclic dinucleotides (CDNs) product of cGAS upon sensing DNA, to prime B cells for IL-27 induction by CD154. Furthermore, B cell-specific deficiency in IL-27 (but not IL-35) or STING in DSS-treated mice resulted in specific impairment in generating plasma cells and T-bet⁺NCR⁺ Group 3 innate lymphoid cells (ILC3s), reduced IgA secretion into the gut lumen and expression of anti-inflammatory IL-22 (and concomitant increase in pro-inflammatory IL-17, as produced by T-bet⁻NCR⁻ILC3s) and severe colitis-like symptoms. These, together with the ability of IL-27-producing B cells generated in vitro by cGAMP priming and CD154 stimulation to prevent lethality caused by sustained DSS treatment, show that B cells in the gut respond to immunoactive CDNs to induce IL-27 for the maintenance of intestinal homeostasis (Illustrated in FIG. 1).

IL-27-producing B cells (B-27 cells) can be produced in T-independent manner. To stimulate mouse B cells with cGAMP for 4 h, wash off, and then add different TLR ligands (TLR1/2 ligand Pam₃CSK₄, TLR4 ligand lipid A, TLR7 ligand R-848, TLR9 ligand CpG) for 24, 48 and 72 h. Programming can be confirmed by measuring transcripts by qRT-PCR and secreted cytokines by ELISA.

B-27 cells produced using methods above are different with respect to the transcriptome from B-27 cells generated in a T cell-dependent manner. To stimulate mouse B cells with cGAMP for 4 h, wash off, and then add TLR9 ligand CpG. For comparison, B cells will be primed with cGAMP or LPS and then stimulated with an anti-CD40 agonistic antibody (which mimicks CD154) plus IL-21 (T cell-dependent regimen).

Human naïve B cells can be programmed to become B-27 cells. Human naïve B cells can be isolated from buffy coat and stimulated with the cGAMP/CpG regimen (T-independent) or the CpG priming/CD154 plus IL-21 regimen (T-dependent).

To analyze engraftment of B-27 cells, Tg(Aicda-cre) Rosa 26-tdTomato transgenic mice will be generated. Spleen B cells can be isolated from Tg(Aicda-cre) Rosa 26-tdTomato mice, and stimulated in vitro with the T-dependent regimen (LPS priming plus anti-CD40 and IL-21) for 48 h to generate programmed B-27 cells.

A cohort of C57 mice can be treated with nil (PBS) or DSS for 5 days. Programmed B-27 cells can be injected into C57 mice pre-treated with nil or DSS, followed by monitoring of body weight/disease activities, performing whole-body imaging analysis to track tdTomato⁺ B-27 cells, and euthanizing mice at Day 30 to examine the presence of tdTomato⁺ B-27 cells in the gut.

Other studies can be performed to ensure B-27 cells programmed by the T-independent regimen can also home to the injured gut in the same mice. Peripheral blood B cells can be isolated from Tg(Aicda-cre) Rosa 26-tdTomato mice and stimulated in vitro with the T-independent regimen (cGAMP/CpG) for 48 h to generate B-27 cells. The Tg(Aicda-cre) Rosa 26-tdTomato donor mice are allowed to recover. Followed by i.v. injection of programmed B-27 cells into the same Tg(Aicda-cre) Rosa 26-tdTomato donor mice at Day 0, with DSS exposure at Day 1, followed by performing whole-body imaging analysis to track tdTomato⁺ B-27 cells and euthanizing mice at Day 7 to examine the presence of tdTomato⁺ B-27 cells in the gut.

To address whether programmed B-27 cells can be cryo-preserved to maintain their viability and function, B cells can be isolated from the spleen of C57 mice, stimulated in vitro with the T-dependent regimen (LPS priming plus anti-CD40 and IL-21) for 48 h to generate programmed B-27 cells. B-27 cells can be cryo-preserve, 5 million cells per vial, 10 vials. After cryo-preservation, B-27 can be i.v. injected into a cohort of C57 mice at Day 0, followed by DSS exposure at Day 1, monitoring of body weight/disease activities and euthanizing the mice at Day 10 to examine the gut.

### Example 2

### Mechanisms Underlying Immune Homeostasis in the Gut and Development of New Therapeutics for Inflammatory Bowel Disease (IBD)

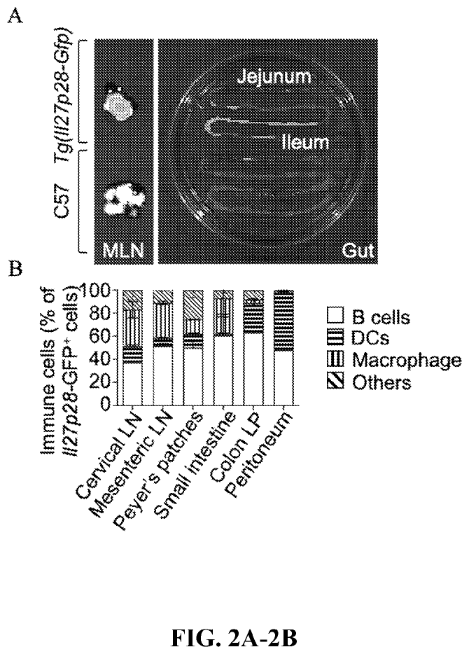

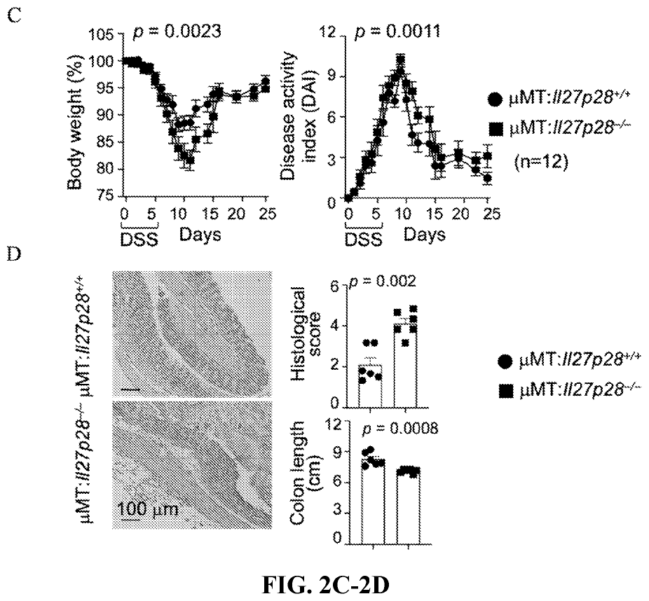

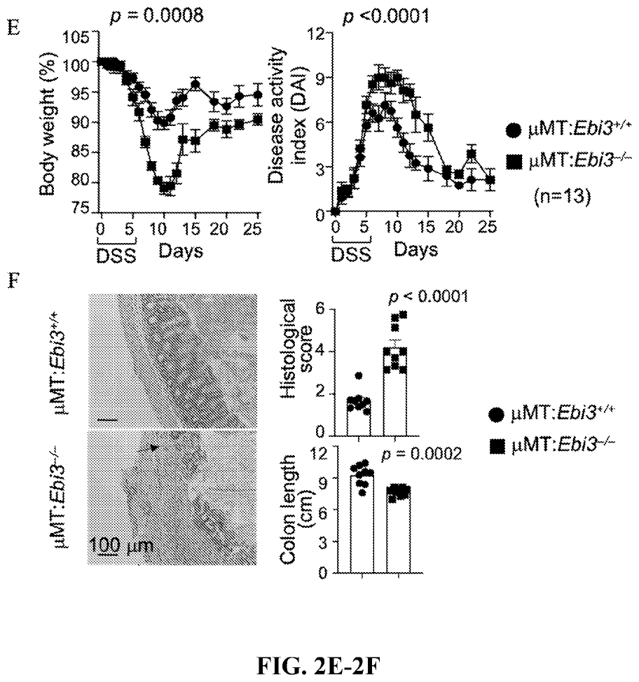

B cells produce pleiotropic cytokine IL-27 that plays an important role in curtailing inflammation in the gut. The concept that B-27 cells exist and regulate a variety of immune functions stems from compelling data generated by our studies on the antibody (Ab) response and acute viral infection (6), Chlamydia immune evasion, breast cancer development, and gut homeostasis—it has been recently expanded by other groups to neuroinflammation (27) and chronic virus infection (28). The novel concept is a significant shift from the paradigm since the discovery of IL-27p28 that myeloid cells are the primary IL-27-producing cells (29). As shown by preliminary data, Il27p28 gene transcription, as quantified by GFP signals from Tg(Il27p28-Gfp) reporter mice (30), was active in the small intestine (SI) in homeostasis (FIG. 2A) and in the entire gut upon treatment with DSS (2% in drinking water for 7 d), with majority of GFP signals segregating within CD19+CD11c−CD11b−CD3−B cells (FIG. 2B). In μMT:Il27p28−/− mice, as generated by mixed (80:20) bone marrow chimera approach involving B cell-null μMT mice and Il27p28−/− mice, the B cell-specific deficiency in Il27p28 resulted in increased susceptibility to DSS-induced colitis, as indicated by heightened body weight loss and disease activities (FIG. 2C). These were associated with more pronounced colonic shortening and mucosal damages, including loss of goblet cells and crypts, lymphocyte infiltration into mucosal and submucosal areas, generation of abscesses, extensive ulceration, and muscle wall thickening (FIG. 2D). The severe colitis in μMT:Il27p28−/− mice was phenocopied in μMT:Ebi3−/− mice (FIG. 2E, 2F), in which B cell expression of EBI3 (the other IL-27 subunit) was ablated. By contrast, μMT:Il12a−/− mice, which lacked B cells that produce IL-35 (IL-12α/EBI3 heterodimer), displayed similar colitis as their μMT:Il12a+/+ mouse counterparts (not shown). Thus, B cell-produced IL-27, but not IL-27p28 alone (or in association with other cytokine subunits) or IL-35, plays an important role in maintaining the gut integrity.

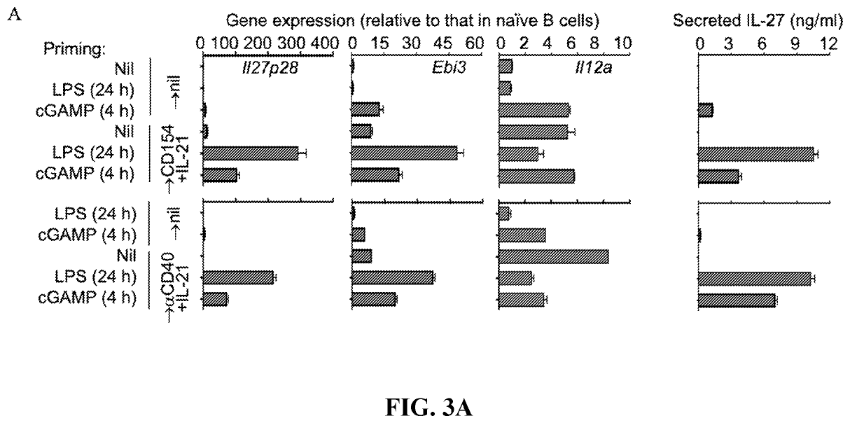

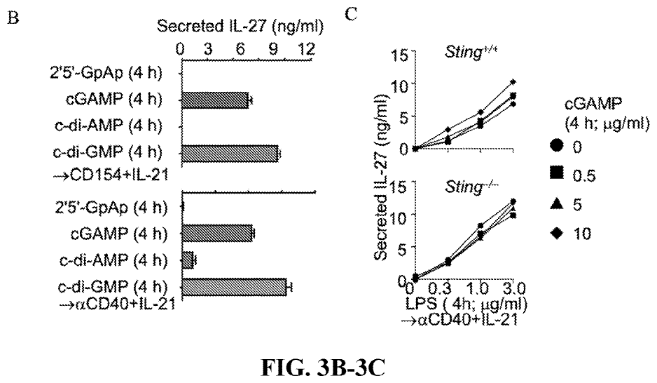

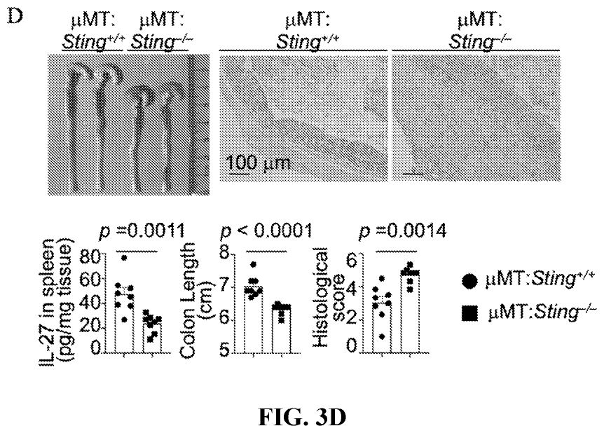

Gut B-27 cells are induced by active CDNs and in a manner dependent on signal adaptor STING. As shown (6), B cell IL-27 expression is induced by TLR ligand priming followed by exposure to CD154 or an agonistic anti-CD40 Ab (αCD40) plus IL-21 (our “gold-standard” condition) at high levels (FIG. 3A), which were comparable to those induced in bone marrow-derived macrophages. This suggests an important role of B cell-intrinsic TLR signaling in B cell priming for B-27 induction in the gut. In addition, other non-TLR innate signals would also play a major role in priming B cells for B-27 cell induction in the gut. As shown by preliminary data, treatment with cGAMP or c-di-GMP (30 μg/ml) for 4 h was sufficient to primer B cells to be exposed to CD154 or αCD40 plus IL-21 for induction of Il27p28 and Ebi3, leading to IL-27 secretion at levels at the same magnitude as those of IL-27 induced by the gold-standard condition (LPS priming and then CD154 or αCD40 plus IL-21; FIG. 3A, 3B). By contrast, IL-27 induction was inefficient when B cells were primed with linear GMP-AMP (2′5′-GpAp), which does not activate STING, or c-di-AMP, which has a much lower affinity to STING than c-di-GMP and rather activates ER adaptor protein (ERAdP) (31) (FIG. 3B). This, together with the additive effect of suboptimal cGAMP and LPS in priming B cells for αCD40/IL-21 induction of IL-27 in Sting+/+ B cells, but not B cells from Stinggt/gt mice (JAX #017537), strongly suggests a B cell-intrinsic role of STING in B-27 cell production (FIG. 3C). Indeed, increased gut damages occurred in DSS-treated μMT:Stinggt/gt chimera mice, in which B cell-specific deficiency in Sting resulted in reduced IL-27 levels (FIG. 3D). The increased sensitivity of μMT:Stinggt/gt mice was similar to that of constitutive KO Stinggt/gt mice, as shown in two studies. μMT:Stinggt/gt mice might have a similar microbiota.

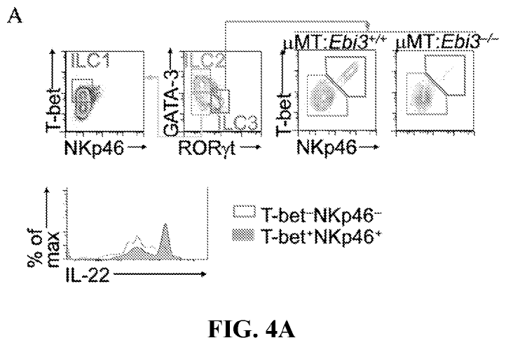

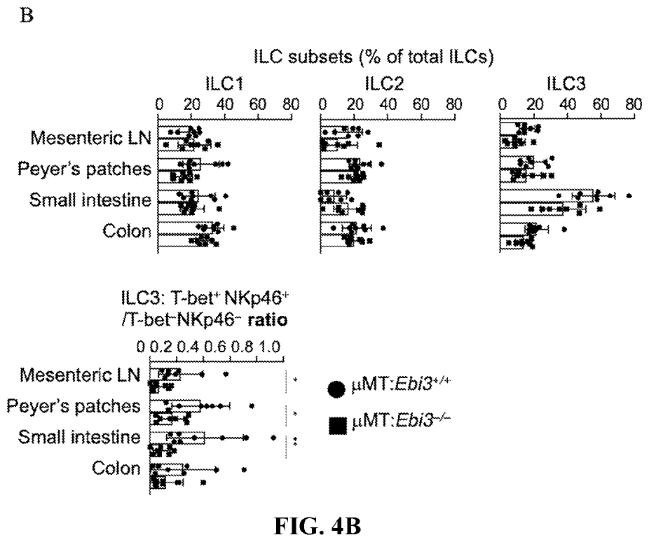

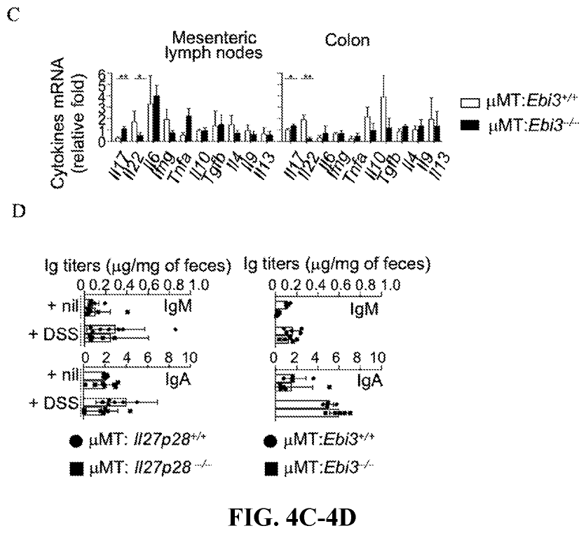

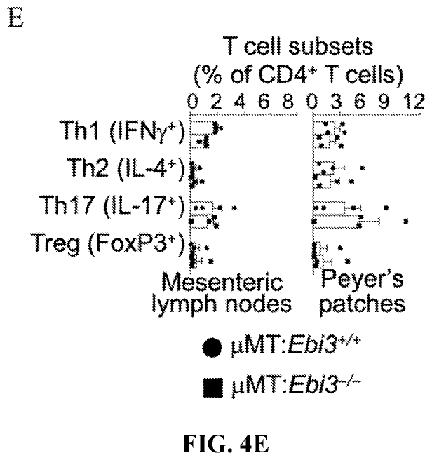

B-27 cells promote ILC3 inter-conversion towards the generation of IL-22-expressing T-bet+NTKp46+ ILC3s. ILCs are a family of innate lymphocytes enriched in barrier tissues, such as the intestine. They orchestrate local tissue homeostasis, but can also contribute to tissue inflammation if dysregulated (4,5). ILCs express little surface markers that define other immune cell lineages (lineage negative, Lin−), but express CD45 and lymphocyte markers IL-7R and CD90. Based on their developmental and functional analogy to CD4+ T cell subsets, ILCs are grouped into three major subsets (FIG. 4A): ILC1s (hallmark TF: T-bet) comprising NK cells and IFNγ-producing ILCs, ILC2s (TF: GATA3) expressing Th2 cytokine IL-5 and IL-13, and ILC3s (TF: RORγt) expressing mixed pro-inflammatory IL-17 and anti-inflammatory IL-22 in a context-dependent manner.

Concomitant with more severe colitis in μMT:Ebi3−/− mice upon DSS treatment, the proportions of T-bet+NKp46+ cells within the Lin−IL-7Rα+RORγt+GATA3−ILC3 compartment were reduced in GALTs, while the proportions of total ILC3s as well as ILC1s and ILC2s were comparable (FIG. 4A, 4B). T-bet+NKp46+ ILC3s preferential produced IL-22 (FIG. 4A), which promotes epithelial cell repair and modulation of T cells to dampen inflammation. By contrast, T-bet¬−/loNKp46−/lo ILC3s produced low levels of protective IL-22, but high levels of IL-17 that would worsen gut damages. As the total number of ILC3s did not change in μMT:Ebi3−/− mice, the imbalance between T-bet+NKp46+ and T-bet¬−/loNKp46−/lo ILC3s and their different preferences in cytokine production are consistent with decreased Il22 and increased Il17 expression (FIG. 4C). Finally, μMT:Ebi3−/− mice were normal in IgM, IgA and (low) IgG titers (FIG. 4D) and proportions of CD4+ T cell subsets in the gut (FIG. 4E), emphasizing that the ILC3 imbalance is likely responsible for the increase in colitis.

### Methods and Materials

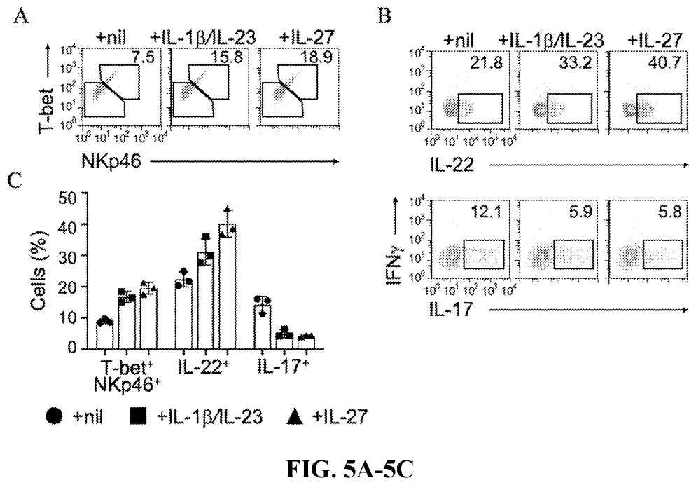

Analyze the impact of recombinant IL-27 (in vitro) and B-27 cells (in vivo) on ILC3 gene protein expression. Tg(Rorc-cre)34 (JAX #022791) and Rosa26fl-STOP-fl-EYFP (JAX #006148) mice will be crossed to generate TgRorc-creRosa26fl-STOP-fl-EYFP mice, which will allow us to sort purify EYFP+IL-7R+CD45+Lin−CD117+ST2−ILC3s. Sorted ILC3s will be cultured in the presence of IL-7 and IL-2, which promote ILC survival, and stimulated them with recombinant IL-27 for 0 h, 6 h, 12 h and 24 h. The proportions of T-bet+NKp46+ and T-bet¬−/loNKp46−/lo ILC3s will be analyzed by FACS, as well as STAT1 and STAT3 phosphorylation (which indicates IL27-triggered STAT activation) and expression of IL-22 and IL-17—The inventors have used the same recombinant IL-27 to induce STAT1/STAT3 activation, T-bet expression, and IFNγ gene signature in B cells (6). Experiments involving enriched and then in vitro amplified ILC3s from C57 mice, show IL-27 upregulated expression of T-bet and IL-22, but reduced IL-17 and did not affect IFNγ (FIG. 5).

Analysis of heightened inflammation in mice in which ILC3s cannot respond to IL-27 and normalize the gut integrity in these mice as well as B27-KO mice by using IL-27-conditioned IL-22-expressing ILC3s. Rorc^{cre/+} mice will be crossed with Il27rafl/fl mice to generate Rorc^{cre/+}Il27rad^{fl/fl} mice, in which IL-27 receptor will be ablated only in cells expressing RORγt, the hallmark TF of ILC3s. The loss of T-bet+NKp46+ and gain of T-bet−/loNKp46−/lo, reduced IL-22 production and increased gut immunopathology will be assessed, but normal T cell subsets in these mice after DSS treatment (n=6), similar to μMT:Ebi3−/− mice (FIG. 4). TNBS-treated mice will be used to confirm findings.

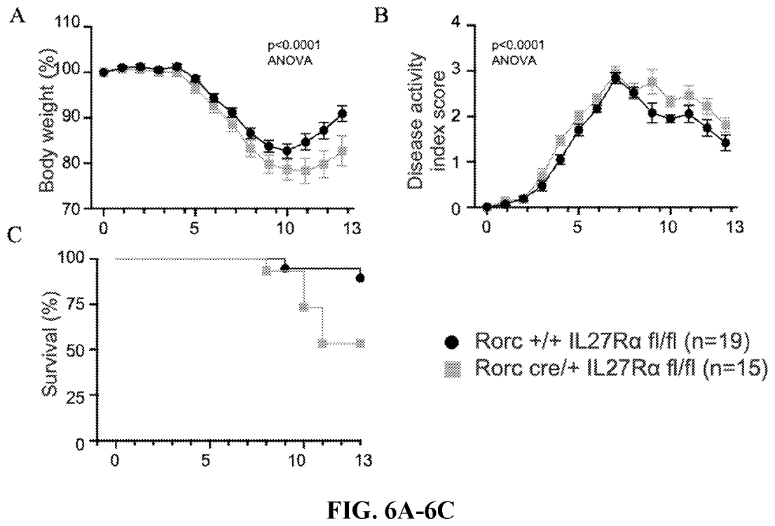

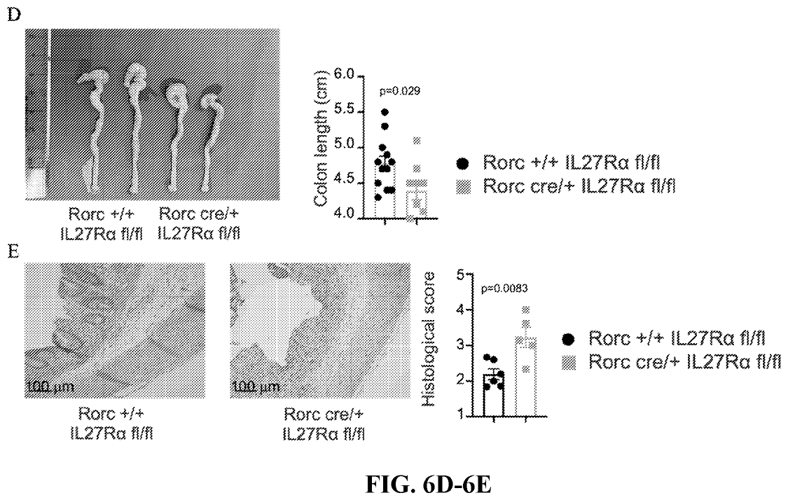

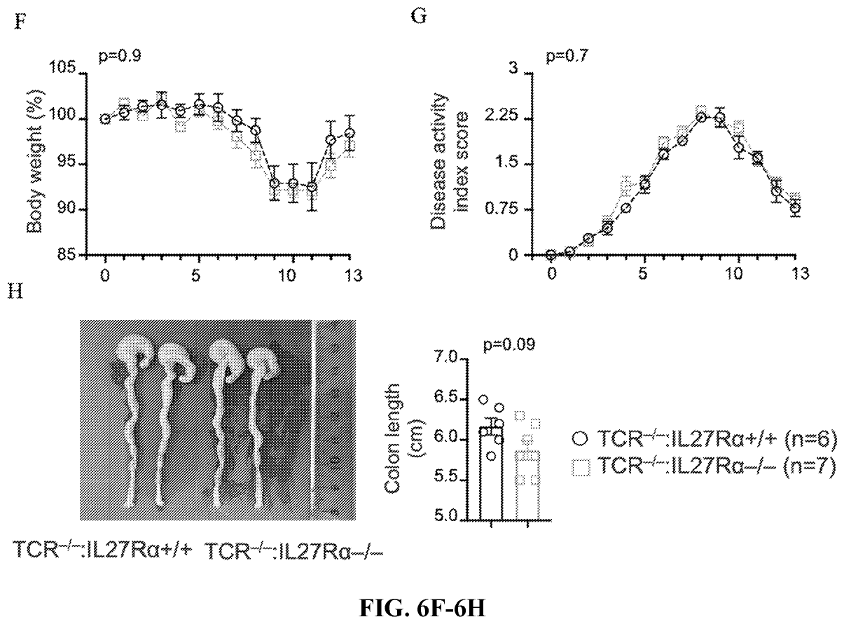

As suggested by the change of ILC3s in mice lacking B-27 cells, as summarized above, B-27 cells would primarily target ILC3s through the IL-27 receptor, which is the heterodimer of IL-27Rα and GP130. Mice with IL-27Rα-deficiency specifically in ILC3s and/or Th17 (Rorc^{cre/+}Il27ra^{fl/fl}) showed more gut damages upon DSS treatment, leading to more disease symptoms, body weight loss and death, as compared to their wildtype littermates (FIG. 6A-6E). However, IL-27R-deficiency specifically in T cells in mixed bone marrow chimera mice (Tcr^{-/-}:Il27ra^{-/-}) did not lead to any alterations in gut inflammation (FIG. 6F-6H). Taken together, IL-27R in ILC3s, but not T cells (including Th17 cells), plays a major role in responding to B-27 cells to maintain the gut homeostasis.

To address the impact of B-27 cells on the interconversion of the two ILC3 subsets in vivo, B27-KO mice were generated by crossing Aicdacre mice (JAX #007700; Aicda is strictly expressed in activated B cells) with Il27p28fl/fl mice. ILC3s and other immune cell populations will be analyzed by FACS (as in FIG. 4) in four gut tissues (MLN, PP, SI LP and colon LP) from DSS-treated mice (n=10/group). As ILCs are maintained locally, the location/proximity of B-27 cells and ILC3s will be analyzed by fluorescence microscopy. The inventors will also use mice treated with gut damage-inducing 2,4,6-trinitrobenzene sulfonic acid (TNBS) as another model.

To gain deep mechanistic insights into how B-27 cells polarize ILC3s, single-cell RNA-Seq (scRNA-Seq, Fluidigm/NextSeq Platform) will be used to analyze total ILCs (IL-7R+CD45+Lin−) sorted from DSS-treated B27-KO and wildtype littermates (n=3). To determine each ILC's transcriptome and, therefore, its identity and characteristics, differentially expressed genes will be analyzed using HiSat2 followed by DESeq, edgeR and CuffLinks, use rtsne and mcluster to assign and quantify the number of total, T-bet+NKp46+ and T-bet¬−/loNKp46−/lo ILC3s and their Il22 expression, GSEA for gene signatures, and MARIna to detect activities of STATs and other TFs.

To address the contribution of IL-27-polarized ILC3s and IL-22 upregulation to gut homeostasis, EYFP+IL-7R+CD45+Lin−CD117+ST2-ILC3s will be sorted from MLNs and SI of TgRorc-creRosa26fl-STOP-fl-EYFP mice, and culture them with IL-2/IL-7 plus IL-1β/IL-23 to amplify ILC3s, resulting in 3×10⁵ ILC3s per donor. ILC3s will be conditioned with IL-27 for 12 h to promote T-bet+NKp46+ ILC3 generation and IL-22 production (FIG. 5). IL-27− or nil-conditioned ILC3s will be intravenously transferred into Rorc^{cre/+}Il27ra^{fl/fl} mice, B27-KO mice and their respective wildtype littermates (n=12), with 10⁴ cells at d−1, d 1 and d3 of DSS treatment. IL-22 levels and colitis will be assessed.

Male and female mice will be used for all in vivo experiments and follow basic principles of pre-clinical studies, including double-blind for pathology scoring and pre-set primary end-points, and avoid ad hoc data exclusion. The inventors have performed power analysis to determine mouse numbers sufficient to detect significant results (p<0.05).

B27-KO mice are expected to have fewer “protective” T-bet+NKp46+ ILC3s and more “pathogenic” T-bet¬−/loNKp46−/lo ILC3s. The inventors are aware that ILCs may be outnumbered by “corresponding” Th cell subsets. Thus, alterations in ILC3s would provide a parsimonious, albeit not full, explanation of expected phenotypes of mutant mice, with the exact contribution of ILC3s addressed by the rescue by IL-27-conditioned ILC3s. IL-27 is expected to upregulate IL-22 in ILC3s and perhaps other genes, as unveiled by scRNA-Seq, which will also extend previous single-cell studies in UC patients (44). IL-27-producing DCs (45,46) or epithelial cells (47) may also play a role, which can be addressed by double or triple KO mice. Colitis symptoms are expected in Tg(Rorc-cre)Il27rafl/fl mice to be reversed by IL-27-conditioned ILC3s.

Testing the prediction that B-27 cells are induced by gut inflammation in humans and humanized H-Mice® and that programmed B-27 cells prevent or alleviate gut injury in wildtype and mutant mice as well as H-Mice®. Occurrence of huB-27 cells will be analyzed in colonoscopy samples from donors with a wide spectrum of gut inflammation and DSS induction of huB-27 cells in H-Mice®.

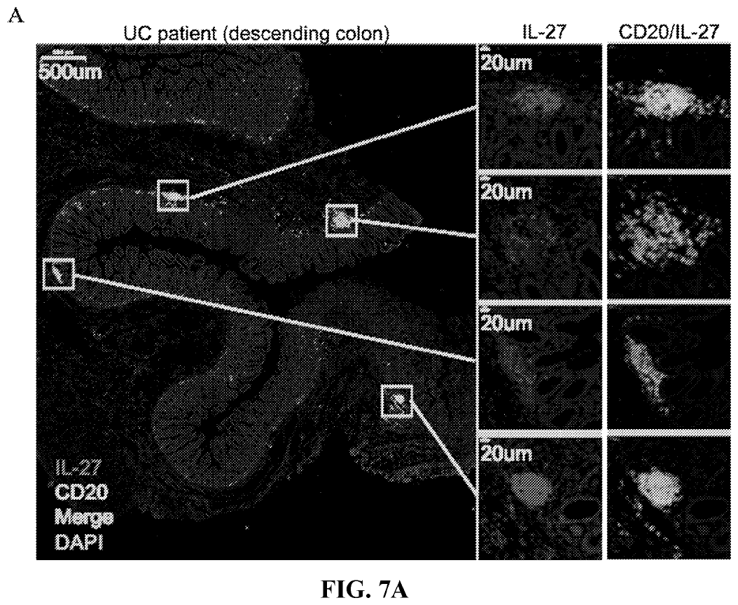

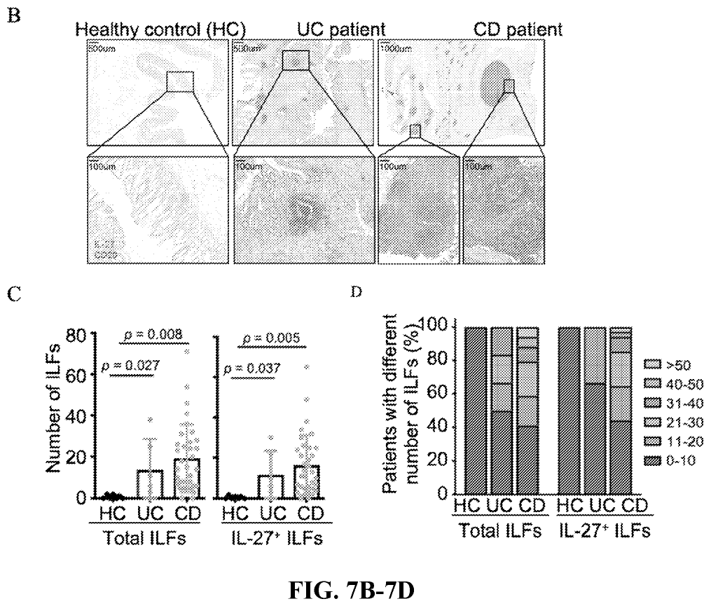

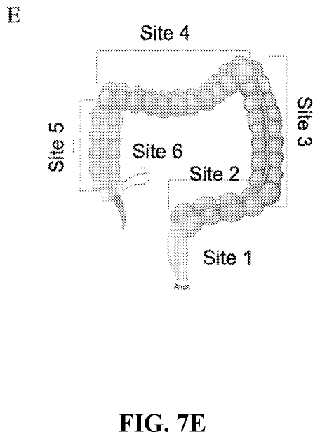

ILFs formed in UC and CD patients and contained CD20+ B cells, many of which expressed IL-27 (FIG. 7A, 7B). The numbers of total ILFs and IL-27+ ILFs were greatly increased in IBD patients (FIG. 7C, 7D). To further establish the correlation of human B-27 (huB-27) cell/ILF induction with inflammation, a two-prong strategy will be employed: (i) by analyzing fresh colonoscopy biopsy samples from 5 groups of donors (18-70-yr old) covering a wide spectrum of inflammation (Table 1), i.e., healthy control (HC) volunteers (Grp 1, N1=20), chronic UC (Grp 2, N2=10) and CD (Grp 3, N3=10) patients in endoscopic remission or with mild endoscopic activities, showing only isolated inflammation pockets (as diagnosed by histology), after treatment (two post-treatment time points, total T=3), and UC patients with acute inflammation in more than two segments (Grp 4, N4=5) and CD patients with acute inflammation (Grp 5, N5=5) who require treatment (three time points, total T=4); (ii) by analyzing biopsy samples in five separate locations for UC (L=5, Site 1-5, FIG. 7E) and six locations for CD (L=6, Site 1-6) from the same subject, as we reason that the inflammation state differs in different locations, with huB-27 cells occurring at higher frequencies in areas with more severe inflammation. Endoscopic activities (Mayo endoscopy score for UC and SES-CD for CD) will be used together with histology to score inflammation. The correlation of huB-27 cells with disease index (Mayo partial score for UC and CDAI or HBI for CD) will also be analyzed. The inventors will perform: (i) flow cytometry to analyze huB-27 cells and other immune cells; (ii) immunofluorescence (as in FIG. 7A) and immunohistochemistry (FIG. 7B) to analyze ILFs, IL-27 and B cell marker CD20 proteins; (iii) RNAScope® to analyze IL27p28 and CD20 transcripts; (iv) scRNA-Seq of (patient/location) samples showing high abundance of huB-27 cells and corresponding HC samples to characterize B cells and other cells.

TABLE 1
Donor/patient recruitment by Dr. J. Echavarma (total 50 donors, 18-70 years
old; 100 colonoscopy visits in total, ~1 visti/wk; samples in 5 or 6 sites.

| Group 1 | Group 2 | Group 3 | Group 4 | Group 5 |
|---|---|---|---|---|
| Healthy Male | UC in remission, | CD in remission, | UC acute | CD acute |
| | male/female | male/female | inflammation, | inflammation, |
| | | | male/female | male/femlae |
| N₁ = 20; | N₂ = 10; | N₃ = 10; | N₄ = 5; | N₅ = 5; |
| T₁ = 1 (0 mo.); | T₂ = 1 (0 and 12 | T₃ = 2 (0 and 12 | T₄ = 4 (0, 1, 3 | T₅ = 4 (0, 1, 3 |
| L₁ = 6 sites | mo); | mo); | and 12 mo); | and 12 mo); |
| | L₂ = 5 sites (1-5) | L₃ = 6 sites | L₄ = 5 sites (1-5) | L₅ = 6 sites |
| Total 120 | Total 100 | Total 120 | Total 100 | Total 120 |
| samples (yr 1-2) | samples (yr 1-2) | samples (yr 1-2) | samples (yr 1-2) | samples (yr 1-2) |

To further prove that inflammation induces huB-27 cells, H-Mice® will be used, an advanced humanized mouse platform owing to the following modifications: use of NSGW41 mice instead of NSG mice as recipients, freshly prepared CD34+ hematopoietic stem cells (HSCs) from cord blood samples from nearby hospitals instead of commercial ones, intracardiac injection of HSCs into 2-day old pups instead of i.v. or intra-hepatic injection, and hormonal conditioning of adult mice. H-Mice® have fully developed secondary lymphoid organs (such as lymph nodes, and mature immune cell compartments, could mount high-affinity and class-switched Ab responses to immunization and flagellin vaccination, resulting in neutralization activities indistinguishable from HC serum samples. Importantly, H-Mice® responded to DSS treatment, as C57 mice do, first losing body weight until d 10 or 12 and then starting to recover.

H-Mice® mice (n=10/group) will be treated with DSS (1% or 2% in drinking water; 7 d) and monitor body weight and colitis DAI daily between d 1 and d 15 and every three days up to d 30. Mice (6/group) will be sacrificed at d 5 to score colon damage in three locations (proximal to rectum, middle and distal). Three samples will be collected, colon lamina propria, small intestine and mesenteric lymph nodes (as in FIG. 2B) for: (i) intracellular staining/FACS and immunofluorescence microscopy to analyze huB-27 cell numbers and location; (ii) cytokine gene expression by qRT-PCR; and (iii) FACS to analyze T cells and ILCs.

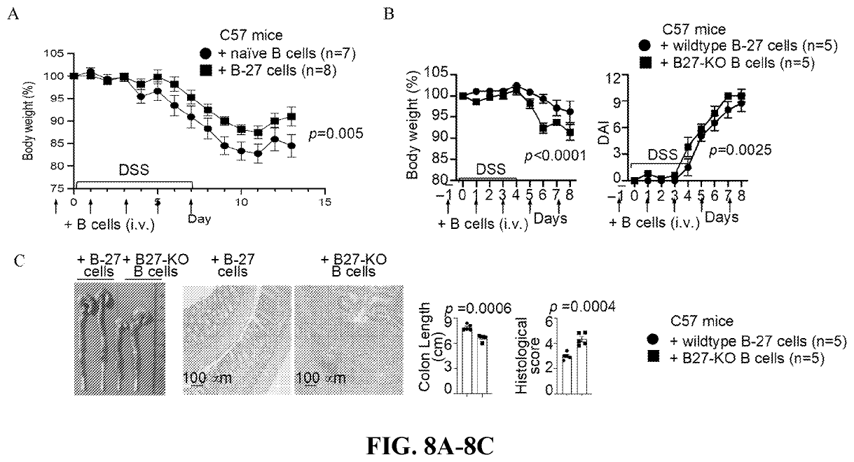

Program murine and human naïve B cells to generate B-27 cells to prevent and/or treat gut inflammation. The inventors further reason that at the sites where tissues are severely damaged (such as those in patients with acute inflammation) due to genetic and/or environmental factors, endogenously induced huB-27 cells may be insufficient for full repair. As such, supplement with programmed B-27 cells may alleviate inflammation in mutant mice and perhaps even abolish the temporary weight loss and diseases in wildtype C57 mice and H-Mice®. Towards this, B cells will be stimulated with LPS/CD154/IL-21 for 48 h (the gold-standard condition for murine B-27 cell induction in vitro, >95%) and inject 10⁶ such programmed B-27 cells i.v., every other day starting at d−1 of DSS treatment, in B-27-KO mice, μMT/Sting−/− mice and their respective wildtype littermates (n=10). As shown by preliminary data, such B-27 cells reduce body weight loss in DSS-treated C57 mice (FIG. 8A). The overall wellness and normalization of the gut and two ILC3 subsets (using FACS and scRNA-Seq) will be analyzed. As comparison to B-27 cells, recombinant IL-27 will be used through i.p. injection (100 ng every other day). To ensure that the rescue effect of programmed B-27 cells is indeed through IL-27, B-27-KO B cell counterparts will be used as negative controls. As shown in preliminary data, DSS-treated C57 mice displayed less severe body weight loss and gut damages when receiving B-27 cells, as compared to those receiving B-27-KO cells (FIG. 8B, 8C).

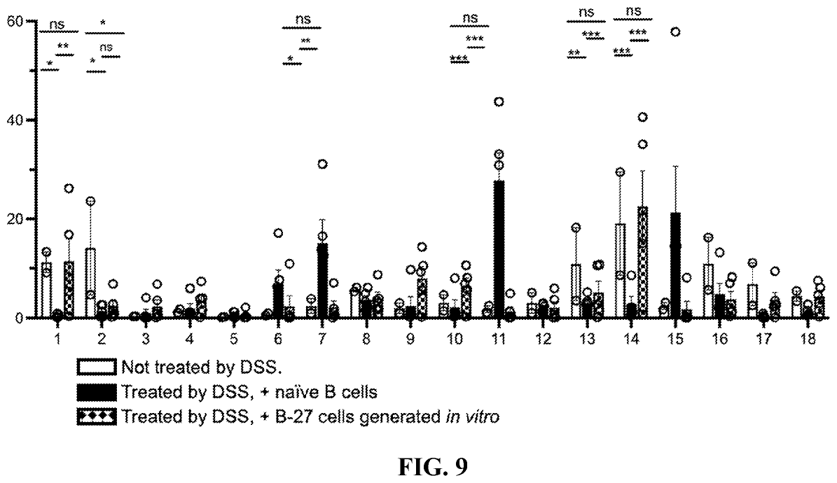

In conjunction with their effect in alleviating DSS-triggered gut damages, B-27 cells could re-normalized the immune landscape in the gut of DSS-treated mice (FIG. 9). Additional analyses will be conducted to analyze the immunomodulatory function of B-27 cells. Importantly, whether B-27 cells lost their therapeutic benefits in Rorc^{cre/+}Il27ra^{fl/fl} mice to test whether B-27 cells need to target IL-27R on (RORgt+) ILC3s to function.

Human naïve B cells, as isolated from buffy coat (South Texas Blood and Tissue Center), will be stimulated with TLR9 ligand CpG (human B cells express no TLR4) plus CD154 and IL-21 to yield B-27 cells. Programmed huB-27 cells will be transferred (10⁶ per injection every other day) to H-Mice® to alleviate DSS colitis (n=12).

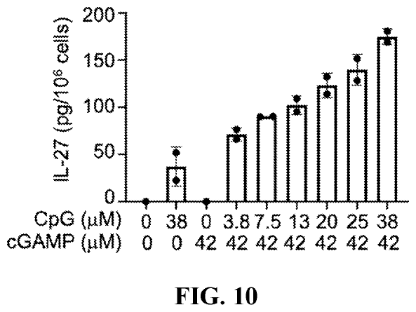

Human naïve B cells, as isolated from buffy coat, will also be stimulated with TLR9 ligand CpG plus cGAMP to yield B-27 cells, which secreted IL-27 (FIG. 10). Programmed huB-27 cells will be transferred (10⁶ per injection every other day) to H-Mice® to alleviate DSS colitis.

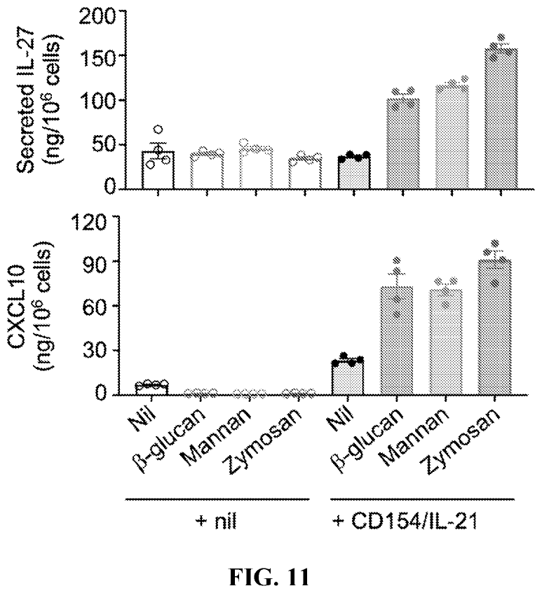

In addition, B-27 cells could be produced in a method completely different from TLR priming or CDN priming described above. Stimulation with Saccharomyces cerevisiae p3-glucan, mannan or zymosan, all of which are cell wall components, followed by stimulation with CD154 and IL-21 also led to robust secretion of IL-27 and CXCL10, a chemokine that is co-produced by B-27 cells (FIG. 11). Thus, B priming with any innate receptors and then stimulation with T cell-derived ligands lead to production of B-27 cells.

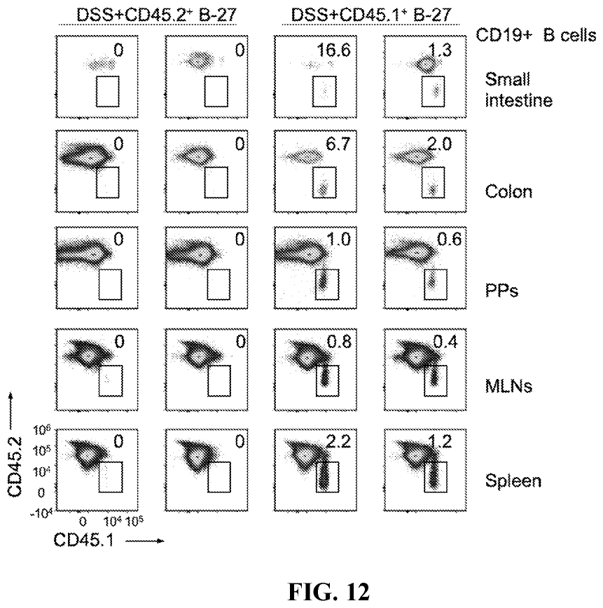

In addition to tracking using fluorescence markers, such as tdTomato in Tg(Aicda-cre) Rosa 26-tdTomato transgenic mice mentioned above, programmed B-27 cells could be tracked using the CD45.1 congenic marker. B-27 cells expressing CD45.1 were injected into C57 mice treated with DSS, followed by monitoring of B-27 cells in the gut tissues and other secondary lymphoid organs. Donor B cells were detected in all of such tissues and organs, albeit with varying frequencies that ranged from 0.43% to 16% (FIG. 12).

### REFERENCES

1. Buonocore et al., Nature 464, 1371-1375 (2010).
2. Wang et al., Cell 171, 201-216 e218 (2017).
3. Mao et al., Nature 554, 255-259 (2018).
4. Pantazi and Powell, Front. Immunol. 10, 676 (2019).
5. Sonnenberg and Artis, Nat. Med. 21, 698-708 (2015).
6. Yan et al., bioRxiv doi. 10.1101/2020.06.26.117010 (2020).
7. Troy et al., J Immunol. 183, 2037-2044 (2009).
8. Sasaoka et al., AJP: Gastrointestinal and Liver Physiology 300, G568-G576 (2011).
9. Tato et al., PLoS One 7, e31680 (2012).
10. Diegelmann et al., J Biol. Chem. 287, 286-298 (2012).
11. Zeng et al., Planta Med. 79, 102-109 (2013).
12. Hanson et al., Gastroenterology 146, 210-221 e213 (2014).
13. Zhu et al., J. Leukoc. Biol. 100, 403-411 (2016).
14. Do et al., Mucosal Immunol. 9, 137-145 (2016).
15. Cui et al., Oncoimmunology 6, e1268309 (2017).
16. McLean et al., Inflamm. Bowel Dis. 23, 1983-1995 (2017).
17. Wang et al., J. Infect. Dis. 217, 198-207 (2018).
18. Rhee et al., Nat. Rev. Gastroenterol. Hepatol. 6, 306-314 (2009).
19. Izcue et al., Annu. Rev. Immunol. 27, 313-338 (2009).
20. Kamada et al., Nat. Rev. Immunol. 13, 321-335 (2013).
21. Yissachar et al., Cell 168, 1135-1148 e1112 (2017).
22. Brown et al., Annu. Rev. Immunol. 37, 599-624 (2019).
23. Furusawa et al., Nature 504, 446-450 (2013).
24. Arpaia et al., Nature 504, 451-455 (2013).
25. Singh et al., Immunity 40, 128-139 (2014).
26. Iyer et al., Cell 173, 1123-1134 e1111 (2018).
27. Choi et al., Proc. Natl. Acad. Sci. U.S.A. 118 (2021).
28. Pratumchai et al., Proc. Natl. Acad. Sci. U.S.A. 119 (2022).
29. Pflanz et al., Immunity 16, 779-790 (2002).
30. Kilgore et al., Immunohorizons 2, 1-11 (2018).
31. Xia et al., Nat. Immunol. 19, 141-150 (2018).
32. Canesso et al., Mucosal Immunol. 11, 820-834 (2018).
33. Ahn et al., Cell Rep. 21, 3873-3884 (2017).
34. Eberl and Littman, Science 305, 248-251 (2004).
35. von Burg et al., Proc. Natl. Acad. Sci. U.S.A. 111, 12835-12840 (2014).
36. Teunissen et al., J Invest. Dermatol. 134, 2351-2360 (2014).
37. Giacomin et al., J. Exp. Med. 212, 1513-1528 (2015).
38. Zhang et al., Hepatology 57, 1620-1631 (2013).
39. Wang et al., Immunity 32, 403-413 (2010).
40. Tumanov et al., Cell Host Microbe 10, 44-53 (2011).
41. Kruglov et al., Science 342, 1243-1246 (2013).
42. Koroleva et al., J. Immunol. Methods 421, 61-72 (2015).
43. Do et al., Proc. Natl. Acad. Sci. U.S.A. 114, 10190-10195 (2017).
44. Boland et al., Sci. Immunol. 5 (2020).
45. Gulden et al., JCI Insight 2 (2017).
46. Lavoie et al., Gastroenterology 158, 1359-1372 e1359 (2020).
47. Lin et al., J. Exp. Med. 218 (2021).
48. Bashford-Rogers et al., Nature 574, 122-126 (2019).
49. Grootjans et al., Science 363, 993-998 (2019).
50. Lund, Curr. Opin. Immunol. 20, 332-338 (2008).
51. Tedder, J. Immunol. 194, 1395-1401 (2015).
52. Shen and Fillatreau, Nat. Rev. Immunol. 15, 441-451 (2015).
53. Ray et al., Int. Immunol. 27, 531-536 (2015).
54. Rosser and Mauri, Immunity 42, 607-612 (2015).
55. Fischbach et al., Sci. Transl. Med. 5, 179ps177 (2013).

1. A method for treating inflammatory bowel disease (IBD) comprising:
(i) obtaining naïve B cells from a compatible subject;
(ii) culturing the naïve B cells in the presence of a toll-like receptor (TLR) agonist and cyclic dinucleotide or a mimic thereof resulting in an IL-27 secreting B cell (B-27 cell); and
(iii) administering the B27 cell to a subject having IBD or suspected of having IBD.

2. The method of claim 1, wherein the naïve B cells are isolated from the subject to be treated.

3. The method of claim 1, wherein the naïve B cells are isolated from the blood.

4. The method of claim 1, wherein the naïve B cells are isolated from peripheral blood mononuclear cells (PBMC).

5. The method of claim 1, wherein the TLR agonist is a TLR 1/2, TLR 4, TLR 7, TLR 9 agonist or a combination thereof.

6. The method of claim 5, wherein the TLR 1/2 agonist is PAM2CSK₄.

7. The method of claim 5, wherein the TLR 4 agonist is lipid A.

8. The method of claim 5, wherein the TLR 7 agonist is R-848.

9. The method of claim 5, wherein the TLR 9 agonist is a CpG oligodeoxynucleotide (ODN).

10. The method of claim 9, wherein the TLR 9 agonist is an ODN having the nucleotide sequence of 5′-tccatgacgttcctgacgtt-3′ (SEQ ID NO:1) or 5′-tcgtcgttttgtcgttttgtcgtt-3′ (SEQ ID NO:2).

11. The method of claim 1, wherein 1×10⁴ to 1×10⁸ B-27 cells are administered.

12. The method of claim 1, wherein the B-27 cells are administered intravenously.

13. A programmed B cell comprising an IL-27 secreting B cell produced by exposing a naïve B cell to IL-27 programming conditions, the programming conditions consisting essentially of exposure to a toll-like receptor (TLR) agonist and cyclic dinucleotide.

14. The programmed B cell of claim 13, wherein the cyclic dinucleotide is cGAMP.

15. The programmed B cell of claim 13, wherein the TLR agonist is CpG oligodeoxynucleotide or lipopolysaccharide (LPS).
